(19) United States
(12) Patent Application Publication
(10) Pub. No.: US 2010/0150960 A1
(43) Pub. Date: Jun. 17, 2010

(54) COMPOSITIONS AND METHODS FOR CHITOSAN ENHANCED IMMUNE RESPONSE

(75) Inventors: Jeffrey Schlom, Potomac, MD (US); David A. Zaharoff, Fayetteville, AR (US); John W. Greiner, Ijamsville, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: The United States of America, as represented by the Secretary,Department of Health and Human Servi, BETHESDA, MD (US)

(21) Appl. No.: 12/442,483

(22) PCT Filed: Sep. 21, 2007
(86) PCT No.: PCT/US07/20540

§ 371 Date: Feb. 22, 2010

Related U.S. Application Data
(60) Provisional application No. 60/846,481, filed on Sep. 22, 2006.

(51) Int. Cl. A61K 39/21; A61K 39/00; A61P 31/12; A61K 39/145; A61K 39/29; A61K 39/015; A61K 38/19; A61P 37/04; A61P 31/16; A61P 31/18; A61P 35/00

(52) U.S. Cl. 424/208.1; 424/184.1; 424/209.1; 424/225.1; 424/272.1; 514/12; 977/773

(57) ABSTRACT

The present invention features methods and compositions related to chitosan antigen depots, and chitosan cytokine depots, and the use of depot compositions in treating and preventing diseases.

### RELATED APPLICATIONS

This application claims the benefit of U.S. Provisional Application No. 60/846,481, filed Sep. 22, 2006. The entire contents of the aforementioned application are hereby incorporated by reference.

### BACKGROUND OF THE INVENTION

Aluminum compounds were the first described adjuvants over 80 years ago. Since that time, over 100 empirically-derived adjuvants and adjuvant variations have been tested both preclinically and clinically (Vogel and Powell. 1995 Pharm Biotechnol 6:141-228). Nearly all of these adjuvants failed to win approval for use in routine vaccines due to toxicity concerns.

Ideally, an adjuvant would elicit a persistent, high quality immune response to an antigen while being non-toxic, biodegradable, non-immunogenic and chemically defined for reproducible manufacture (Gupta and Siber. 1995 Vaccine 13(14):1263-76).

Over 20 years ago, chitin derivatives, including chitosan, were found to have immunostimulatory activity. This immunostimulatory activity, along with the structural similarities between chitin derivatives and glucans, an immunoadjuvant class of natural polysaccharides, led several scientists to study the adjuvant capabilities of chitosan. Studies with chitosan and its derivatives focused on its affects on the immune response when coupled with other adjuvants. Chitosan was regarded as an immune stimulant, and therefore was never considered as a vaccine delivery system.

Because of its mucoadhesive properties, chitosan has been explored as an adjuvant for mucosal vaccination. The mechanisms of vaccine enhancement by chitosan are believed to be due to both retention of vaccine in the nasal passages via mucoadhesion and opening of endothelial cell junctions for paracellular transport of vaccine (Illum et al. 2001 Adv Drug Deliv Rev 51(1-3):81-96).

Nonetheless, chitosan solution alone has never been tested as a vaccine delivery system or depot for subcutaneous administration. This is most likely due to two reasons. First, the mucoadhesive advantage of chitosan is lost during a non-mucosal administration. Second, the high viscosities of chitosan solutions have been overlooked as a way to control the release of antigens. Chitosan, by virtue of its long polymer backbones, forms a highly viscous solution in mild aqueous solvents, which may be a useful property for the controlled release of agents.

Thus, there is a need in the art to describe and apply the adjuvant characteristics of chitosan solution formulated with a model protein antigen for other routes of vaccination.

### SUMMARY OF THE INVENTION

In preferred aspects, the invention features methods and compositions related to chitosan antigen depots, and chitosan cytokine depots, and the use of depot compositions in treating diseases. The invention also features kits comprising chitosan antigen depot and chitosan cytokine depots. Other features and advantages of the invention will be apparent from the detailed description, and from the claims.

Accordingly, in one aspect, the invention features a method for producing an immune response in a subject, the method comprising the steps of mixing one or more antigens with chitosan, or a derivative thereof, to form a depot, and administering the chitosan antigen depot to the subject, thereby producing an immune response in the subject.

In another particular aspect, the invention describes a method for increasing a cell mediated immune response in a subject, the method comprising the steps of mixing one or more antigens with chitosan, or a derivative thereof, and administering the chitosan antigen depot to the subject thereby increasing the cell mediated immune response in the subject.

In another particular aspect, the invention describes a method for increasing a humoral immune response in a subject, the method comprising the steps of mixing one or more antigens with chitosan, or a derivative thereof, and administering the chitosan antigen depot to the subject thereby increasing the humoral immune response in the subject.

In one preferred embodiment of these methods, the chitosan antigen mixture is administered by one or more routes selected from the group consisting of subcutaneous, intradermal, intramuscular, intratumoral injection and intravesical and transdermal administration.

In another aspect, the invention features a method for treating or preventing human immunodeficiency virus in a subject, the method comprising the step of administering a depot composition comprising one or more HIV antigens and chitosan, or a derivative thereof, to the subject, thereby treating or preventing human immunodeficiency virus. In one embodiment, the HIV antigen is selected from the group consisting of gp120, p24, gp41, p17, HIV gag protein, HIV RT protein, HIV Nef protein, HIV pol protein, HIV env protein, HIV Tat protein.

In another particular aspect, the invention features a method for treating or preventing cancer in a subject, the method comprising the step of administering a depot composition comprising one or more cancer antigens and chitosan, or a derivative thereof, to the subject thereby treating or preventing cancer. In a particular embodiment of the method, the cancer antigen is a tumor associated antigen. In a further embodiment, the cancer antigen is selected from the group consisting of hTERT, HSPs, Her2/neu, progesterone receptors, androgen receptors, normal or mutated EGFR, CEA, MART-1, MAGE-1, MAGE-3, LAGE-1, LAGE-2, BAGE family antigens, XAGE family antigens, GAGE family antigens, GP-100, MUC-1, MUC-2, point mutated ras oncogene, normal or point mutated p53, CA-125, PSA, PSMA, C-erb/B2, BRCA I, BRCA II, tyrosinase, SCP-1, CT-7, TRP-1, TRP-2, NY-ESO-1, NY-BR-1, NY-BR-1-85, NY-BR-62, NY-BR-85, HOXB7, PDEF, HPV E7, TAG72, TALE, KSA, SART-3, MTAs, WT1, Survivin, Mesothelin, bcr-abl, pax3-fkhr, ews-fli-1, Ku70/80, RCAS1, cytokeratins, stathmin, vimentin, tumor-associated antigen (TAA), whole tumor cells, tumor specific antigen, tissue specific antigen, modified TAAs, splice variants of TAAs, functional epitopes and epitope agonists thereof.

In another aspect, the invention features a method for treating or preventing malaria in a subject, the method comprising the step of administering a depot composition comprising one or more malaria antigens and chitosan, or a derivative thereof to the subject, thereby treating or preventing malaria. In a particular embodiment, the malaria antigen is selected from the group consisting of MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa.

A further aspect of the invention features a method for treating or preventing hepatitis in a subject, the method comprising the step of administering a depot composition comprising one or more hepatitis antigens and chitosan, or a derivative thereof to the subject, thereby treating or preventing hepatitis. In a particular preferred embodiment of the method, the one or more hepatitis antigens are selected from the group consisting of formalin-inactivated hepatitis virus, HBsAg, HBeAg, HDAgs, HAV proteins and epitopes, HBV proteins and epitopes, HCV proteins and epitopes, HDV proteins and epitopes, and HEV proteins and epitopes.

Yet a further aspect of the invention features a method for treating or preventing influenza in a subject, the method comprising the steps of administering a depot composition comprising one or more influenza antigens and chitosan, or a derivative thereof to the subject, thereby treating or preventing influenza. In one embodiment of the method, the one or more antigens are selected from the group consisting of HA, NA, H5N1, H1N1, H2N2, H3N2, H7N7, H1N2, H9N2, H7N2, H7N3, H10N7, and HPAI A (H5N1).

Another particular aspect of the invention teaches a method for increasing an immune response in a subject, the method comprising the step of administering a depot composition comprising one or more cytokines and chitosan, or a derivative thereof to a subject, thereby increasing an immune response in a subject.

In one embodiment, the immune response is a cell mediated immune response. In another embodiment, the immune response is a humoral immune response. In still another embodiment of the method, the cytokine is an interleukin. In a particular embodiment, the cytokine is selected from the group consisting of IL-1 alpha, IL-1beta, IL-2, IL-7, IL-10, IL-12, IL-15, IL-18, IL-23 and IL-27. In another particular embodiment, the cytokine is GM-CSF, IFN-alpha, IFN-gamma, IFN-beta, TGF-beta, TNF-alpha, TNF-beta, IL-2, IL-7, IL-12, or IL-15. In a related embodiment, the cytokine is recombinant GM-CSF (rGM-CSF). In still a further embodiment, the cytokine is a chemokine. In a particular embodiment, the chemokine is selected from the group consisting of C, CC, CXC and CX₃C. In another particular embodiment, the chemokine is selected from the group consisting of lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine.

In another embodiment of the methods of the invention, the depot composition is administered by one or more routes selected from the group consisting of subcutaneous, intradermal, intramuscular, intratumoral injection and intravesical and transdermal delivery.

Another particular aspect of the invention teaches a method for increasing an immune response in a subject, the method comprising the step of administering a depot composition comprising one or more cytokines, one or more antigens and chitosan, or a derivative thereof to a subject, thereby increasing an immune response in a subject.

In another aspect, the invention teaches a method for producing an immune response in a subject, the method comprising the steps of mixing nanoparticles, comprised of chitosan or a derivative thereof, containing one or more antigens and delivering the antigen-containing chitosan-based nanoparticles to a subject, thereby producing an immune response in the subject.

In one embodiment of the method, the immune response is a cell mediated immune response. In another embodiment, the immune response is a humoral immune response. In a particular embodiment, the nanoparticle further comprises one or more cytokines.

In another aspect, the invention teaches a method for increasing a cell mediated immune response in a subject, the method comprising the steps of mixing particles, comprised of chitosan or a derivative thereof, containing one or more antigens and delivering the antigen-containing chitosan-based particles to a subject, thereby producing an immune response in the subject.

In one embodiment of the method, the particles are microparticles. In another embodiment, the particles are nanoparticles. In a particular embodiment, the microparticle or nanoparticle is multilayered. In another particular embodiment, the particle contains one or more cytokines. In a particular embodiment of the method, the cytokine is selected from the group consisting of IL-1 alpha, IL-1beta, IL-2, IL-7, IL-10, IL-12, IL-15, IL-18, IL-23, IL-27, GM-CSF, IFN-alpha, IFN-gamma, IFN-beta, TGF-beta, TNF-alpha, TNF-beta, C, CC, CXC, CX₃C, lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine. In a related embodiment, the cytokine is recombinant GM-CSF (rGM-CSF).

Another particular aspect of the invention features a method for treating or preventing human immunodeficiency virus in a subject, the method comprising the steps of mixing nanoparticles or microparticles, comprised of chitosan or a derivative thereof, containing one or more antigens; and delivering the antigen-containing chitosan-based nanoparticles or microparticles to a subject, thereby treating or preventing human immunodeficiency virus in the subject. In a particular embodiment of the method, the HIV antigen is selected from the group consisting of gp120, p24, gp41, p17, HIV gag protein, HIV RT protein, HIV Nef protein, HIV pol protein, HIV env protein, and HIV Tat protein.

A further aspect of the invention features a method for treating or preventing cancer in a subject, the method comprising the steps of mixing nanoparticles or microparticles, comprised of chitosan or a derivative thereof, containing one or more antigens, and delivering the antigen-containing chitosan-based nanoparticles or microparticles to a subject, thereby treating or preventing cancer in the subject. In a particular embodiment of the method, the one or more antigens are tumor associated antigens. In another embodiment of the method, the one or more antigens are selected from the group consisting of hTERT, HSPs, Her2/neu, progesterone receptors, androgen receptors, normal or mutated EGFR, CEA, MART-1, MAGE-1, MAGE-3, LAGE-1, LAGE-2, BAGE family antigens, XAGE family antigens, GAGE family antigens, GP-100, MUC-1, MUC-2, point mutated ras oncogene, normal or point mutated p53, CA-125, PSA, PSMA, C-erb/B2, BRCA I, BRCA II, tyrosinase, SCP-1, CT-7, TRP-1, TRP-2, NY-ESO-1, NY-BR-1, NY-BR-1-85, NY-BR-62, NY-BR-85, HOXB7, PDEF, HPV E7, TAG72, TAL6, KSA, SART-3, MTAs, WT1, Survivin, Mesothelin, bcr-abl, pax3-fkhr, ews-fli-1, Ku70/80, RCAS1, cytokeratins, stathmin, vimentin, tumor-associated antigen (TAA), tumor specific antigen, whole tumor cells, tissue specific antigen, modified TAAs, splice variants of TAAs, functional epitopes and epitope agonists thereof.

Another particular aspect of the invention features a method for treating or preventing malaria in a subject, the method comprising the steps of mixing nanoparticles or microparticles, comprised of chitosan or a derivative thereof, containing one or more antigens, and delivering the antigen-containing chitosan-based nanoparticles or microparticles to a subject, thereby treating or preventing malaria in the subject. In a particular embodiment of the method, the one or more antigens are selected from the group consisting of MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa.

In another aspect, the invention features a method for treating or preventing hepatitis in a subject, the method comprising the steps of mixing nanoparticles or microparticles, comprised of chitosan or a derivative thereof, containing one or more antigens, and delivering the antigen-containing chitosan-based nanoparticles or microparticles to a subject, thereby treating or preventing hepatitis in the subject. In a particular embodiment of the method, the one or more antigens are selected from the group consisting of formalin-inactivated hepatitis virus, HBsAg, HBeAg, HDAgs, HAV proteins and epitopes, HBV proteins and epitopes, HCV proteins and epitopes, HDV proteins and epitopes, HEV proteins and epitopes.

In a further particular aspect, the invention features a method for treating or preventing influenza in a subject, the method comprising the step of mixing nanoparticles or microparticles, comprised of chitosan or a derivative thereof, containing one or more antigens, and delivering the antigen-containing chitosan-based nanoparticles or microparticles to a subject, thereby treating or preventing influenza in the subject. In a particular embodiment of the method, the one or more antigens are selected from the group consisting of HA, NA, H5N1, H1N1, H2N2, H3N2, H7N7, H1N2, H9N2, H7N2, H7N3, H10N7, and HPAI A (H5N1).

In a further embodiment of the methods of the invention the chitosan nanoparticles or microparticles are delivered by one or more routes selected from the group consisting of subcutaneous, intradermal, intramuscular, intratumoral injection and intravesical and transdermal delivery.

Another particular aspect of the invention teaches a method for increasing cell mediated immune response in a subject, the method comprising the steps of mixing one or more antigens with chitosan, or a derivative thereof administering the chitosan antigen mixture to a subject, and administering one or more cytokines to the subject, thereby increasing cell mediated immune response in the subject.

In one embodiment, the method further comprises the step of administration of one or more additional vaccines in the subject. In a particular embodiment, administration of the one or more additional vaccines occurs before administration of the chitosan antigen mixture. In a further particular embodiment of the method, administration of the additional one or more additional vaccines occurs after administration of the chitosan antigen mixture. In another embodiment of the method, administration of the additional one or more additional vaccines occurs concurrently with administration of the chitosan antigen mixture.

In another aspect, the invention teaches a method for increasing cell mediated immune response in a subject, the method comprising the steps of mixing one or more cytokines with chitosan, or a derivative thereof, administering the chitosan cytokine mixture to a subject, administering one or more additional vaccines in the subject, and thereby increasing cell mediated immune response in the subject.

In one embodiment of the method, the cytokine is selected from the group consisting of GM-CSF, IL-2, IL-7, IL-12, IL-15, IL-18, IL-23, IL-27, IFN-gamma, and IFN-alpha. In a related embodiment, the cytokine is recombinant GM-CSF (rGM-CSF). In a further embodiment of the method, the cytokine is administered before administration of the one or more additional vaccines. In a further particular embodiment, the cytokine is administered after administration of the one or more additional vaccines. In still another particular embodiment of the method, the cytokine is administered by one or more routes selected from subcutaneous, intradermal, intramuscular, intratumoral injection and intravesical and transdermal delivery.

In a further aspect, the invention teaches a method for treating or preventing cancer in a subject, the method comprising the step of administering a depot composition comprising one or more small molecule inhibitors and chitosan, or a derivative thereof, to the subject, and thereby treating or preventing cancer.

In one embodiment, the small molecule inhibitor is selected from the group consisting of: siRNA, shRNA, DNA aptamers, RNA aptamers, and antisense oligonucleotides.

In another aspect, the invention features a method for treating or preventing cancer in a subject, the method comprising the step of administering a depot composition comprising one or antibodies, or fragments thereof, and chitosan, or a derivative thereof, to the subject; thereby treating or preventing cancer.

In a particular embodiment of the method, the antibody, or fragment thereof, is selected from the group consisting of monoclonal or polyclonal antibodies.

In another embodiment, the methods of the invention are used for prophylactic treatment. In a further embodiment, the methods of the invention are used for therapeutic treatment.

Another particular aspect of the invention features a depot composition for administration in a subject, the depot composition comprising one or more antigens and chitosan, or a derivative thereof.

A further particular aspect of the invention features a depot composition for administration in a subject, the depot composition comprising one or more cytokines and chitosan, or a derivative thereof.

In one embodiment, the cytokine is selected from the group consisting of IL-1 alpha, IL-1beta, IL-2, IL-7, IL-10, IL-12, IL-15, IL-18, IL-23, IL-27, GM-CSF, IFN-alpha, IFN-gamma, IFN-beta, TGF-beta, TNF-alpha, TNF-beta, C, CC, CXC, CX₃C, lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine. In a related embodiment, the cytokine is recombinant GM-CSF (rGM-CSF). In a further embodiment, the depot composition is a vaccine depot composition. In another particular embodiment, the antigen is selected from the group consisting of virus-encoding antigen, inactivated virus, replication-defective virus, protein, yeast constructs containing antigen, antibody, anti-idiotypic antibody, lipid, carbohydrate, cell, cell extract, and cell fragment.

In a further aspect, the invention features a vaccine depot composition for administration to a subject for the treatment or prevention of human immunodeficiency virus, the depot composition comprising one or more HIV antigens and chitosan, or a derivative thereof.

In one embodiment, the antigen is selected from the group consisting of gp120, p24, gp41, p17, HIV gag protein, HIV RT protein, HIV Nef protein, HIV pol protein, HIV env protein, HIV Tat protein.

Another aspect of the invention features a vaccine depot composition for administration to a subject for the treatment or prevention of cancer, the depot composition comprising one or more cancer antigens and chitosan, or a derivative thereof.

In a particular embodiment, the one or more cancer antigen is a tumor associate antigen. In a further embodiment, the antigen is selected from the group consisting of hTERT, HSPs, Her2/neu, progesterone receptors, androgen receptors, normal or mutated EGFR, CEA, MART-1, MAGE-1, MAGE-3, LAGE-1, LAGE-2, BAGE family antigens, XAGE family antigens, GAGE family antigens, GP-100, MUC-1, MUC-2, point mutated ras oncogene, normal or point mutated p53, CA-125, PSA, PSMA, C-erb/B2, BRCA I, BRCA II, tyrosinase, SCP-1, CT-7, TRP-1, TRP-2, NY-ESO-1, NY-BR-1, NY-BR-1-85, NY-BR-62, NY-BR-85, HOXB7, PDEF, HPV E7, TAG72, TAL6, KSA, SART-3, MTAs, WT1, Survivin, Mesothelin, bcr-abl, pax3-fkhr, ews-fli-1, Ku70/80, RCAS1, cytokeratins, stathmin, vimentin, tumor-associated antigen (TAA), tumor specific antigen, whole tumor cells, tissue specific antigen, modified TAAs, splice variants of TAAs, functional epitopes and epitope agonists thereof.

Another aspect of the invention features a vaccine depot composition for administration to a subject for the treatment or prevention of cancer, the depot composition comprising one or more small molecule inhibitors and chitosan, or a derivative thereof.

In one embodiment, the small molecule inhibitor is selected from the group consisting of siRNA, shRNA, DNA aptamers, RNA aptamers, and antisense oligonucleotides.

A further aspect of the invention features a vaccine depot composition for administration to a subject for the treatment or prevention of cancer, the depot composition comprising one or more antibodies, or fragments thereof, and chitosan, or a derivative thereof.

In one embodiment, the antibody, or fragment thereof, is selected from the group consisting of monoclonal or polyclonal antibodies.

Another aspect of the invention features a vaccine depot composition for administration to a subject for the treatment or prevention of malaria, the depot composition comprising one or more malaria antigens and chitosan, or a derivative thereof.

In a particular embodiment, the antigen is selected from the group consisting of MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa.

Another aspect of the invention features a vaccine depot composition for administration to a subject for the treatment or prevention of hepatitis, the depot composition comprising one or more hepatitis antigens and chitosan, or a derivative thereof.

In one embodiment, the antigen is selected from the group consisting of formalin-inactivated hepatitis virus, HBsAg, HBeAg, HDAgs, HAV proteins and epitopes, HBV proteins and epitopes, HCV proteins and epitopes, HDV proteins and epitopes, and HEV proteins and epitopes.

A further aspect of the invention features a vaccine depot composition for administration to a subject for the treatment or prevention of influenza, the depot composition comprising one or more influenza antigens and chitosan, or a derivative thereof.

In a particular embodiment, the antigen is selected from the group consisting of HA, NA, H5N1, H1N1, H2N2, H3N2, H7N7, H1N2, H9N2, H7N2, H7N3, H10N7, and HPAI A (H5N1).

Another aspect of the invention features a vaccine depot composition for administration to a subject for the enhancement of an immune response, the depot composition comprising one or more cytokines and chitosan, or a derivative thereof.

In one embodiment the cytokine is a chemokine. In a further embodiment, the chemokine is selected from the group consisting of: C, CC, CXC, CX₃C, lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine.

In a particular embodiment, the chitosan, or derivative thereof, is selected from the group consisting of Carboxymethyl-, Hydroxyethyl-, Dihydroxypropyl-, Acetyl-, Phosphorylated-, Sulphonated-, N-acetyl-, N-proprionyl-, N-butyryl-, N-pentanoyl- and N-hexanoyl-, glycol-chitosans. In another particular embodiment of the aspects, the chitosan, or derivative thereof, is deacetylated. In one embodiment, the chitosan, or derivative thereof, is at least 30% deacetylated. In another embodiment of the aspect, the chitosan, or derivative thereof, is at least 50% deacetylated. In one particular embodiment, the chitosan, or derivative thereof, is at least 70% deacetylated.

In further embodiments, the depot composition of the aspects of the invention features chitosan, or derivative thereof, that is high molecular weight chitosan. In a related embodiment, the chitosan, or derivative thereof, is ≧100 kDa. In another embodiment, the chitosan, or derivative thereof, is low molecular weight chitosan. In a related embodiment, the chitosan, or derivative thereof, is <100 kDa. In further embodiments, the depot composition of the aspects of the invention features the chitosan, or derivative thereof, that is a chitosan salt. In a related embodiment, the chitosan salt is selected from the group consisting of chitosan hydrochloride, chitosan hydroglutamate, chitosan hydrolactate. In another embodiment, the chitosan, or derivative thereof, is modified by chemical crosslinking. In a related embodiment, the chemical crosslinking is to an agent selected from the group consisting of dialdehydes, citric acid, methacrylic acid, lactic acid, or alginate. In another embodiment, the chitosan, or derivative thereof, is modified by redox gelation. In a related embodiment, the redox gelation is carried out with ammonium persulfate and N,N,N′,N′-tetramethylethelynediamine. In another embodiment, the chitosan, or derivative thereof, is formulated with polyol salts to form a hydrogel. In a related embodiment, the polyol salts are selected from the group consisting of glycerol-, sorbitol-, fructose- and glucose-phosphate salts.

In further embodiments of the depot composition of the aspects of the invention, the chitosan is administered in a concentration ranging from 0.1 to 5.0% weight/volume. In still further embodiments, the weight:weight ratio of antigen to chitosan is in the range of 1:3 to 1:100. In further embodiments of the depot composition of the aspects of the invention, the composition further comprises an additional adjuvant. In related embodiments, the adjuvant is selected from the group consisting of CpG motifs, Imiquimod, LPS, MPL, MF59, Ribi Detox™, Alum, QS-21, Freund's complete adjuvant, Freund's incomplete adjuvant, MDP, TDM, ISCOMS, Adjuvant 65, Lipovant, TiterMax®, Montanide ISA720, BCG, Levamisole, squalene, Pluronic, Tween 80, inulin, polyinosinic-polycytidylic acid or any other TLR ligand.

In particular embodiment, the antigen and chitosan, or derivative thereof, are mixed. In still further embodiments, the composition is administered by one or more routes selected from the group consisting of subcutaneous, intradermal, intramuscular, intratumoral injection and intravesical and transdermal delivery.

Another aspect of the invention features a kit comprising a chitosan antigen depot, together with instructions for use.

A further aspect of the invention features a kit comprising a chitosan cytokine depot, together with instructions for use.

In one embodiment, the kit comprises a chitosan antigen cytokine depot, together with instructions for use. In a related embodiment, the cytokine is selected from the group consisting of IL-1alpha, IL-1beta, IL-2, IL-7, IL-10, IL-12, IL-15, IL-18, IL-23, IL-27, GM-CSF, IFN-alpha, IFN-gamma, IFN-beta, TGF-beta, TNF-alpha, TNF-beta. In an alternative embodiment, the cytokine is recombinant GM-CSF (rGM-CSF). In a further embodiment, the cytokine is a chemokine. In a related embodiment, the chemokine is selected from the group consisting of C, CC, CXC, CX₃C, lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine.

Another aspect of the invention features a kit for increasing the efficacy of a vaccine comprising chitosan and instructions for use.

A further aspect of the invention features a method for making a vaccine depot composition comprising mixing chitosan, or a derivative thereof, with one or more antigens, thereby making a vaccine depot composition.

Another aspect of the invention features a method for making a vaccine depot composition comprising mixing chitosan, or a derivative thereof, with one or more antigens, in the presence of a buffer or solvent, thereby making a vaccine depot composition.

A further aspect of the invention features a method for making a cytokine depot composition comprising mixing chitosan, or a derivative thereof, with one or more cytokines, thereby making a cytokine depot composition.

Still a further aspect of the invention features a method for making a cytokine depot composition comprising mixing chitosan, or a derivative thereof, with one or more cytokines, in the presence of a buffer or solvent, thereby making a cytokine depot composition

In one embodiment, the buffer or solvent is selected from the group consisting of water, deionized water, PBS, DPBS, HBSS, HEPES, ethanol, methanol, acetic acid, hydrochloric acid, sodium hydroxide solution. In another embodiment, the chitosan is used at a concentration of 0.1 to 5.0% weight/volume. In a further embodiment, the method further comprises the step of adjusting the pH. In a particular embodiment, the pH is in the range of 3.0 to 9.0.

### BRIEF DESCRIPTION OF THE DRAWINGS

FIG. 1 is a graph demonstrating that chitosan enhanced antigen-specific CD4⁺ proliferation, with beta-galactosidase (β-gal)-vaccinated mice showing a robust enhancement in antigen-specific CD4⁺ proliferation. Splenic CD4⁺ proliferative responses from C57BL/6 mice (n=3) vaccinated with 100 μg beta-galactosidase (β-gal) in PBS () or 1.5% chitosan (▪) were assessed 1 week after the booster vaccination, and assessment involved culture of 150,000 CD4⁺ cells from experimental animals with 500,000 irradiated antigen presenting cells from unvaccinated control mice in the presence of increasing concentrations of β-gal. Data are presented as mean±SEM and representative of two independent experiments, wherein an asterisk (*) denotes a P value of less than 0.05. Chitosan did not affect the quality of CD4⁺ splenocytes as judged by the CD4⁺ response to a non-specific T cell mitogen, concanavalin A (refer to insert).

FIGS. 2a to 2c present three panels of graphs demonstrating that chitosan enhanced antigen-specific serum IgG. FIG. 2a shows that chitosan enhanced the antigen-specific antibody titer in the linear portion of a titration (O.D.=1.0) approximately 5.3-fold when beta-galactosidase-specific serum IgG from C57BL/6 mice (n=3) vaccinated with 100 μg beta-galactosidase (β-gal) in PBS () or 1.5% chitosan (▪) were measured 1 week after the booster vaccination via ELISA. There were no differences in serum IgG against control antigen (ovalbumin; refer to insert). FIGS. 2b and 2c show that chitosan enhanced antigen-specific IgG₁ and IgG₂ₐ titers 5.9- and 8.0-fold, respectively, at O.D.=1.0, likely reflecting a mixed T_{H}1/T_{H}2 response, when beta-galactosidase (β-gal)-specific serum IgG₁ and IgG₂ₐ, respectively, from vaccinated mice were measured 1 week after the booster vaccination via ELISA. All data are represented as mean±S.E.M, and are representative of two independent experiments (n=3). All increases in antibody titer approximated at an optical density of 1.0 were statistically significant (P<0.001).

FIG. 3 is a graph demonstrating that chitosan elicited a robust DTH response. Delayed-type hypersensitivity responses in C57BL/6 mice (n=4) primed and boosted with 100 μg beta-galactosidase (β-gal) in PBS () or 1.5% chitosan (▪) were measured 1 week after the booster vaccination. 50 μg beta-galactosidase (β-gal) in 10 μl PBS were injected into the pinnae of vaccinated mice. Opposite pinnae were injected with 10 μl PBS. Ear thickness was measured 24 h after ear injections. The thickness of the ear challenged with antigen was divided by the thickness of the ear challenged with PBS to obtain percent increase in ear thickness. Ear swelling was significantly greater (P<0.01) in mice vaccinated with beta-galactosidase (β-gal) in chitosan.

FIGS. 4a and 4b present two graphs showing that chitosan was equipotent to IFA as a subcutaneous vaccine adjuvant. FIG. 4a shows splenic CD4⁺ proliferative responses in C57BL/6 mice (n=3) vaccinated with 100 μg β-gal in 1.5% chitosan (▪) or IFA () and FIG. 4b shows beta-galactosidase (β-gal) specific serum IgG from such mice, assessed 1 week after the booster vaccination. 100,000 CD4⁺ cells from experimental animals were cultured with 500,000 irradiated antigen presenting cells from unvaccinated control mice in the presence of increasing concentrations of β-gal. Proliferative responses were indistinguishable (P>0.1). Data are represented as mean±SEM.

FIGS. 5a and 5b show two graphs demonstrating that chitosan was superior to aluminum hydroxide as a subcutaneous vaccine adjuvant. FIG. 5a shows splenic CD4⁺ proliferative responses in C57BL/6 mice (n=3) vaccinated with 100 μg β-gal in PBS () or 1.5% chitosan (▪) or aluminum hydroxide (▴) and FIG. 5b shows beta-galactosidase (β-gal)-specific serum IgG from such mice, assessed 1 week after the booster vaccination. 200,000 CD4⁺ cells from experimental animals were cultured with 500,000 irradiated antigen presenting cells from unvaccinated control mice in the presence of increasing concentrations of β-gal. Chitosan outperformed aluminum hydroxide in enhancing antigen-specific CD4⁺ proliferation and serum IgG titers. Chitosan increased antigen-specific antibody titer 6.6-fold over aluminum hydroxide at an optical density of 1.0. Data are represented as mean±SEM. An asterisk (*) indicates observation of a P value of less than 0.05 versus aluminum hydroxide.

FIG. 6 is a panel of ten fluorescence images showing that chitosan maintained a depot of β-galactosidase (β-gal). Spatiotemporal distributions of a single subcutaneous administration of a fluorescently-labeled model antigen (Alexa Fluor 660-labeled β-galactosidase) were acquired via non-invasive fluorescence imaging. Fluorescence intensity was used as surrogate for β-gal concentration. The region of interest used to quantify fluorescence intensity is denoted by a circle.

FIG. 7 is a graph demonstrating dissipation of a model antigen (beta-galactosidase (β-gal)) from a subcutaneous injection site. C57BL/6 mice were shaved and treated with a depilatory cream prior to injection of Alexa Fluor 660-labeled β-gal in PBS () or 1.5% chitosan (▪). The fluorescence intensity of Alexa Fluor 660-labeled β-gal in a region of interest around the injection site was used as a surrogate of β-gal concentration. Within 24 h, less than 3% of the antigen delivered in PBS remained at the injection site. Greater than 60% of antigen delivered in chitosan remained 7 days after injection. Data are represented as mean±SEM from four mice per group.

FIGS. 8a to 8c present three panels of hematoxylin and eosin (H & E) staining of the subcutis, demonstrating that subcutaneous chitosan depot was infiltrated and degraded in 2-3 weeks: FIG. 8a shows H&E staining of the subcutis at 2 days after a subcutaneous injection of 1.5% chitosan, while FIGS. 8b and 8c show such staining at 7 and 14 days, respectively.

FIG. 9 shows that rGM-CSF disseminated much more slowly from an injection site if administered in a solution of chitosan, thereby demonstrating that a chitosan solution maintained a depot of rGM-CSF. Spatiotemporal distributions of a single subcutaneous administration of Alexa Fluor 660-labeled rGM-CSF were acquired via non-invasive fluorescence imaging, and fluorescence intensity was used as surrogate for rGM-CSF concentration. The region of interest used to quantify fluorescence intensity is denoted by a circle.

FIG. 10 shows the differing rates of dissemination of rGM-CSF from a subcutaneous injection site when administered in PBS (rGM-CSF was undetectable in 12 to 24 hrs) or if formulated with chitosan solution (measured for 9 to 10 days). C57BL/6 mice were shaved and treated with a depilatory cream prior to injection of Alexa Fluor 660-labeled rGM-CSF in PBS (), 1% chitosan solution (▪) or 2% chitosan solution. The fluorescence intensity of Alexa Fluor 660-labeled rGM-CSF in a region of interest around the injection site was used as a surrogate of rGM-CSF concentration. Data are represented as mean±SEM from four mice per group.

FIGS. 11a to 11c show that chitosan/rGM-CSF outperformed rGM-CSF alone in antigen presenting ability of draining lymph nodes, with cells from mice treated with chitosan/rGM-CSF observed to generate greater allogeneic T cell proliferation than cells from mice treated with rGM-CSF alone. FIG. 11a shows results obtained for draining inguinal lymph nodes from C57BL/6 mice (n=5) injected once subcutaneously with PBS () or chitosan/rGM-CSF (20 μg) (▴), or given 4 daily s.c. injections of rGM-CSF (▪) and harvested at 7 days after the initial injection, while FIG. 11b and FIG. 11e show such results at 14 days and 35 days after the initial injection, respectively. Lymph node cells were irradiated and co-incubated with decreasing concentration of allogeneic (Balb/c) T cells. Responses were transient and returned to control levels within 35 days. Data are represented as mean±SEM. An asterisk (*) denotes observation of a P value less than 0.05 compared to PBS, while a double asterisk (**) denotes a P value less than 0.05 compared to rGM-CSF alone.

FIG. 12 shows that chitosan/rGM-CSF enhanced antigen-specific CD4⁺ proliferation better than either adjuvant alone. Splenic CD4⁺ proliferative responses from C57BL/6 mice (n=4) vaccinated with 5 μg UV-inactivated influenza in PBS (), chitosan solution (♦), rGM-CSF (with 3 additional daily vaccinations) (▪), chitosan/rGM-CSF (20 μg) (▴), or chitosan/rGM-CSF (80 μg) (Δ) were assessed 1 week after the booster vaccination. Two hundred-thousand CD4⁺ cells from experimental animals were cultured with 500,000 irradiated antigen presenting cells from unvaccinated control mice in the presence of increasing concentrations of UV-inactivated influenza. Mice vaccinated with antigen in chitosan/rGM-CSF demonstrated a robust enhancement in antigen-specific CD4⁺ proliferation that was better than either adjuvant alone. Proliferative responses from all of the adjuvants were statistically greater than no adjuvant (PBS) (P<0.5 for the three highest concentrations of UV-inactivated influenza). Proliferative responses from chitosan/rGM-CSF (20 μg) and chitosan/rGM-CSF (80 μg) were statistically greater than either chitosan or rGM-CSF alone (P<0.05) but were indistinguishable from each other (P>0.05). Data are represented as mean±SEM.

FIG. 13 demonstrates that chitosan/rGM-CSF (20 μg) induced maximal antigen-specific CD4⁺ proliferation. Splenic CD4⁺ proliferative responses from C57BL/6 mice (n=4) vaccinated with 5 μg UV-inactivated influenza in chitosan solution (), chitosan/rGM-CSF (5 μg) (♦), chitosan/rGM-CSF (10 μg) (▪) or chitosan/rGM-CSF (20 μg) (▴) were assessed 1 week after the booster vaccination. Two hundred-thousand CD4⁺ cells from experimental animals were cultured with 500,000 irradiated antigen presenting cells from unvaccinated control mice in the presence of increasing concentrations of UV-inactivated influenza. Chitosan/rGM-CSF (20 μg) outperformed the two lower doses of rGM-CSF in chitosan; however, the three results were statistically indistinguishable (P>0.05). Data are represented as mean±SEM.

FIG. 14 shows that administration of chitosan/rGM-CSF generated greater numbers of peptide-specific CD8⁺ splenocytes following vaccination, with increasing doses of rGM-CSF formulated with chitosan solution resulting in modest increases in peptide-specific CD8⁺ splenocytes. Spleens from C57BL/6 mice (n=4) vaccinated with 5 μg UV-inactivated influenza in chitosan solution, chitosan/rGM-CSF (5 μg), chitosan/rGM-CSF (10 μg) or chitosan/rGM-CSF (20 μg) were assessed 1 week after the booster vaccination. Splenocytes were pooled and stained with pentamer specific for Flu NP₃₆₆₋₃₇₄ peptide. The values on the graphs represent the percentages of CD8⁺ cells that were also pentamer positive. LCMV NP₃₉₆₋₄₀₄ specific pentamer was used as a control and resulted in between 0.3-0.4% pentamer positive staining for all four groups (not shown).

FIG. 15 shows that administration of chitosan/rGM-CSF generated greater numbers of peptide-specific CD8⁺ splenocytes following in vitro stimulation, with both chitosan/rGM-CSF (10 μg) and chitosan/rGM-CSF (20 μg) resulting in nearly one out of every five cells specific for Flu NP₃₆₆₋₃₇₄ peptide. Spleens from vaccinated mice were harvested as before (FIG. 14). Splenocytes were stimulated in tissue culture flasks with 10 ng/ml Flu NP₃₆₆₋₃₇₄ peptide for one week. Cells were then harvested and stained with pentamer as before. The values on the graphs represent the percentages of CD8⁺ cells that were also pentamer positive. The one week in vitro stimulation of splenocytes magnified the number of peptide-specific CD8⁺ splenocytes (vs. FIG. 14). LCMV NP₃₉₆₋₄₀₄ specific pentamer was used as a control and ranged from 3.7 to 4.2% pentamer positive staining for all four groups (not shown).

FIG. 16 shows that administration of chitosan/rGM-CSF (20 μg) induced maximal CTL. Spleens from C57BL/6 mice (n=4) vaccinated with 5 μg UV-inactivated influenza in chitosan solution (), chitosan/rGM-CSF (5 μg) (▪), chitosan/rGM-CSF (10 μg) (♦) or chitosan/rGM-CSF (20 μg) (▴) were harvested 1 week after the booster vaccination. Splenocytes were stimulated in tissue culture flasks with 10 ng/ml Flu NP₃₆₆₋₃₇₄ peptide for one week. Cells were then harvested and assayed for CTL activity against Flu NP₃₆₆₋₃₇₄ peptide loaded EL4 cells via ¹¹¹In release.

FIGS. 17a to 17d show that chitosan nanoparticles manufactured in a range of sizes. FIG. 17a shows an image of FITC-BSA particles of effective diameter of 262.1 nm (bar indicates 5 μm). FIG. 17b shows the lognormal size distribution for this preparation. FIG. 17c shows an image of FITC-BSA particles of effective diameter of 1283.2 nm (bar indicates 5 μm). FIG. 17d shows the lognormal size distribution for this preparation.

FIG. 18 shows phagocytic uptake of FITC-BSA/chitosan nanoparticles by JAWS II cells at 15 minutes and one hour after administration.

FIG. 19 presents flow cytometry analyses showing in vitro uptake of FITC-BSA encapsulated in chitosan nanoparticles into JAWS II cells—a murine immature dendritic cell line.

FIG. 20 presents flow cytometry analyses showing in vitro uptake of FITC-BSA encapsulated in chitosan nanoparticles into bone marrow-derived murine dendritic cells.

FIGS. 21a to 21c present flow cytometry analyses showing in vivo uptake of FITC-BSA encapsulated in chitosan nanoparticles into lymph node cells. FIG. 21a shows results for PBS administration. FIG. 21b shows results for FITC-BSA administration. FIG. 21c shows results for FITC-BSA administration in chitosan nanoparticles.

FIG. 22 shows antigen-specific CD4⁺ responses in mice vaccinated with β-gal antigen in saline (), chitosan solution (▪), and chitosan nanoparticles (▴). The most robust CD4⁺ response was observed for administrations using chitosan nanoparticles.

FIG. 23 shows that chitosan decreased the antigen-specific CD4+ response to a co-formulated vaccine consisting of recombinant fowlpox encoding influenza nucleoprotein and a triad of costimulatory molecules (rF-Flu/TRICOM).

FIG. 24 shows that chitosan increased the antigen-specific CD4+ response to a co-formulated vaccine consisting of recombinant yeast construct containing carcinoembryonic antigen (CEA)

FIGS. 25A-D show the growth of implanted MC32a tumors in mice as a function of time in response to intratumoral injections of (a) PBS, (b) chitosan, (c) rIFN-γ (25 k IU) or (d) chitosan/rIFN-γ (25 k IU) on days 7 and 14.

FIGS. 26A-E show the growth of implanted MC32a tumors in mice as a function of time in response to intratumoral injections of (a) PBS, (b) rIL-12 (1 μg), (c) chitosan, (d) chitosan/rIL-12 (1 μg) and (e) chitosan/rIL12 (5 μg) on days 7, 14, and 21.

FIG. 27 shows the survival of mice given MC32a tumors at day 0 and intratumoral injections of PBS, rIL-12 (1 μg), chitosan, chitosan/rIL-12 (1 μg) or chitosan/rIL12 (5 μg) on days 7, 14, and 21. The survival curves of the latter two groups are overlapping at 100%.

### DETAILED DESCRIPTION OF THE INVENTION

In preferred aspects, the present invention features methods and compositions for enhancing an immune response in a subject. The invention features method of treating diseases with depot compositions as described.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “adjuvant” is meant to refer to a compound, or combination of compounds which, while not having any specific antigenic effect by themself can stimulate or potentiate an immune response. Adjuvants according to the invention include, but are not limited to basic polyamino acid or a mixture of basic polyamino acids, such as apolyarginine, polylysine, orpolyornithine, or histones, protamines, polyethyleneimines or mixtures thereof. The adjuvant may preferably be CpG motifs, Imiquimod, LPS, MPL, MF59, RIBI DETOX™, Alum, QS-21, Freund's complete adjuvant, Freund's incomplete adjuvant, MDP, TDM, ISCOMS, Adjuvant 65, Lipovant, TITERMAX, Montanide ISA720, BCG, Levamisole, squalene, Pluronic, TWEEN, inulin, polyinosinic-polycytidylic acid or any other TLR ligand.

The terms “administration” or “administering” are defined to include an act of providing a compound or pharmaceutical composition of the invention to a subject in need of treatment.

The term “adaptive immune response” refers to an antigen-specific immune response. In an adaptive immune response, the antigen first must be processed and recognized. Once an antigen has been recognized, the adaptive immune system creates an army of immune cells specifically designed to attack that antigen.

The term “antigen” is meant to refer to any substance that causes the immune system to produce antibodies against it. An antigen may be a foreign substance from the environment such as chemicals, bacteria, viruses, or pollen. An antigen may also be formed within the body, as with bacterial toxins or tissue cells. The term is meant to encompass any antigenic or immunogenic polypeptides including poly-aminoacid materials having epitopes or combinations of epitopes, and immunogen-encoding polynucleotides. In addition, the term is also meant to encompass any poly-saccharide material useful in generating immune response.

The term “antigen depot” refers to an antigenic composition that has properties including extended regional antigenic stimulation, slow release of antigen, and long term retention of antigen.

The term “cancer” refers to any disease that is caused by or results in inappropriately high levels of cell division, inappropriately low levels of apoptosis, or both. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “cell-mediated immune response” refers to the activation of macrophages, natural killer cells or the generation of cytotoxic CD8-positive T-cells and CD4-positive helper-T-cells, which bring about destruction of the tumor cells or of the cells attacked by the pathogen.

A “chemokine” is a specific type of cytokine with a conserved cysteine motif and which can serve as an attractant. Chemokines are described in, for example, Roitt, I., Brostoff, J., Male, D. Immunology. Sixth Edition, Mosby, New York, 2001.

A “cytokine” is a generic term for extracellular proteins or peptides that mediate cell-cell communication, often with the effect of altering the activation state of cells. Cytokines are described in, for example, Roitt, I., Brostoff, J., Male, D. Immunology. Sixth Edition, Mosby, New York, 2001.

The term “decreased” means a negative alteration. A decrease can be change that is a 5%, 10%, 20%, 30%, 40%, 50%, 60%, 70%, 80%, 90%, 95% negative alteration. A decrease can be a fold-change, for example 2-fold, 5-fold, 10-fold.

The term “humoral immune response” refers to the production of immunoglobulins which selectively recognize tumor cells or structures derived from pathogens and consequently, together with other systems such as, for example, complement, ADCC (antibody dependent cytotoxicity) or phagocytosis, bring about the destruction of these tumor cells or the cells attacked by the pathogenic agents.

The term “immune response” refers to an antigen-mediated activation of lymphocytes and the subsequent coordination of the immune system to eliminate the antigen and/or its source. Included is the process whereby inflammatory cells are recruited from the blood to lymphoid as well as non-lymphoid tissues via a multifactorial process that involves distinct adhesive and activation steps. Inflammatory conditions cause the release of chemokines and other factors that, by upregulating and activating adhesion molecules on inflammatory cells, promote adhesion, morphological changes, and extravasation concurrent with chemotaxis through the tissues.

The term “increased” means a positive alteration. An increase can be a change that is a 5%, 10%, 20%, 30%, 40%, 50%, 60%, 70%, 80%, 90%, 95% alteration. An increase can be a fold-change, for example a 2-fold, 5-fold, 10-fold alteration.

The phrase “in combination with” is intended to refer to all forms of administration that provide the compounds of the invention together, e.g. antigen, chitosan, or derivative thereof, cytokine, additional adjuvant, and can include sequential administration, in any order.

The term “subject” refers to animals, typically mammalian animals, such as primates (humans, apes, gibbons, chimpanzees, orangutans, macaques), domestic animals (dogs and cats), farm animals (horses, cattle, goats, sheep, pigs) and experimental animals (mouse, rat, rabbit, guinea pig). Subjects include animal disease models (e.g., mouse models).

The term “interleukin” refers to a group of cytokines that are biological response modifiers (substance that can improve the body's natural response to infection and disease) that help the immune system fight infection and cancer. These substances are normally produced by the body. They are also made in the laboratory for use in treating cancer and other diseases. IL-1 through IL-13, IL-17, IL-18, IL-23 have been described. Exemplary interleukins according to the invention include: IL-1alpha, IL-1beta, IL-2, IL-7, IL-10, IL-12, IL-15, IL-18, IL-23 and IL-27.

The terms “treat,” “treating,” “treatment,” and the like are meant to refer to reducing or ameliorating a disorder and/or symptoms associated therewith. It will be appreciated that, although not precluded, treating a disorder or condition does not require that the disorder, condition or symptoms associated therewith be completely eliminated.

The term “tumor” is intended to include an abnormal mass or growth of cells or tissue. A tumor can be benign or malignant.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Chitosan

Chitosan is a nontoxic, biocompatible, biodegradable, natural polysaccharide that is cleared by enzymatic digestion (Hirano et al. 1989 Biomaterials 10(8):574-6; Sashiwa et al. 1990 Int J Biol Macromol 12(5):295-6). Chitosan has been used safely in humans for topical, intranasal and oral applications (Mhurchu et al. 2005 Obes Rev 6(1):35-42; Pittler and Ernst. 2004 Am J Clin Nutr 79(4):529-36; Read et al. 2005 Vaccine 23(35):4367-74; Mills et al. 2003 Infect Immun 71(2):726-32; McNeela et al. 2004 Vaccine 22(8):909-14; Wedmore et al. 2006 J Trauma 60(3):655-8). Chitosan is a linear polysaccharide formed from repeating beta (1-4 linked) N-acetyl-D-glucosamine and D-glucosamine units, and is derived from the partial deacetylation of chitin obtained from the shells of crustaceans. Chitosan is usually made commercially by a heterogeneous alkaline hydrolysis of chitin to give a product which possesses a random distribution of remaining acetyl moieties. The properties of chitosans depend upon inter alia the degree of deacetylation, and the molecular weight. Most commercially available chitosans contain a population of chitosan molecules of varying molecular weights and varying concentrations of the component N-acetyl-D-glucosamine and D-glucosamine groups. The immunological properties of chitosans are known to be linked to the ratio between the N-acetyl-D-glucosamine and D-glucosamine groups. Chitosan, by virtue of its long polymer backbones, forms a highly viscous solution in mild aqueous solvents; a 1% (w/v) chitosan solution is two orders of magnitude more viscous than water. Viscous solutions are widely used for the controlled release of drugs and macromolecules (Einmahl et al. 2001 Adv Drug Deliv Rev 53(1):45-73; MacKenzie et al. 1980 Br J Obstet Gynaecol 1980; 87(4):292-5; Wang et al. 2003 Mol Cancer Ther 2(11):1233-42). In humans, chitosan has been used as a pharmaceutical excipient, a controversial weight loss supplement, an experimental nasal vaccine adjuvant and in an FDA-approved hemostatic dressing.

Over 20 years ago, chitin derivatives, including chitosan, were found to be potent activators of macrophages and NK cells (Nishimura et al. 1984 Vaccine 2(1):93-9; Nishimura et al. 1986 Vaccine 4(3):151-6). This immunostimulating activity along with the structural similarities between chitin derivatives and glucans, an immunoadjuvant class of natural polysaccharides, led several scientists to study the adjuvant capabilities of chitosan. Nishimura et al. formulated various chitin derivatives with antigen and incomplete Freund's adjuvant (IFA) to measure adaptive immune responses (Nishimura et al. 1985 Vaccine 3(5):379-84). Both 70% and 30% deacetylated chitosan, when formulated with IFA, increased antigen-specific serum antibody titers in mice by over 3-fold versus IFA alone. Similarly, in guinea pigs, chitosan plus IFA induced greater DTH responses than IFA alone (ibid.). Marcinkiewicz et al. found that intraperitoneal (i.p.) administration of a water insoluble chitosan suspension enhanced humoral responses but not cell-mediated immune responses in mice (Marcinkiewicz et al. 1991 Arch Immunol Ther Exp (Warsz) 39(1-2):127-32). Subcutaneous administrations of chitosan suspensions were found to be ineffective (ibid.). In other studies, Seferian and Martinez found that chitosan particles, formulated in an emulsion with antigen, squalene and Pluronic® L121, gave a prolonged, high antigen-specific antibody titer and sensitized animals for antigen-specific DTH responses following an i.p. injection (Seferian and Martinez. 2001 Vaccine 19(6):661-8). Chitosan particles alone offered no enhancement of an adaptive immune response (ibid.). In all of the aforementioned studies, chitosan was regarded as an immune stimulant, and therefore, never considered as a subcutaneous or parenteral vaccine delivery system.

The chitosan, or derivative thereof, of the invention is deacetylated. In certain embodiments, the chitosan, or derivative thereof is a partially deacetylated chitosan, which is 10%, 20%, 30%, 40%, 50%, 60%, 70%, 80%, 90%, 95%, 96%, 97%, 98%, 99% or 100% deacetylated. For example, when the chitosan is one which is at least 70% deacetylated, for example 70-80%, more preferably 72-78% deacetylated, particular examples being 73%, 74%, 75%, 76% and 77% deacetylated. In certain embodiments, the weight average molecular weight of the chitosan, or a derivative thereof, is ≧100 kDa. In other particular embodiments, the weight average molecular weight of the chitosan, or derivative thereof, is <100 kDa.

Chitosan nanoparticles can be manufactured in a range of sizes. In certain embodiments, the instant invention employs chitosan that possesses an effective diameter of between 100 nm and 10 μm, between 200 μm and 2 μm, or between 250 nm and 1500 μm. In some embodiments, chitosan particles have an effective diameter of 100-200 nm, 150-250 nm, 200-300 nm, 250-350 nm, 300-400 nm, 350-450 nm, 400-500 nm, 450-550 nm, 500-600 nm, 550-650 nm, 600-700 nm, 650-750 nm, 700-800 nm, 750-850 nm, 800-900 nm, 850-950 nm, 900 nm to 1 μm, 950 nm to 1.05 μm, 1-1.1 μm, 1.05-1.15 μm, 1.1-1.2 μm, 1.15-1.25 μm, 1.2-1.3 μm, 1.25-1.35 μm, 1.3-1.4 μm, 1.35-1.45 μm, 1.4-1.5 μm, 1.45-1.55 μm, 1.5-1.6 μm, 1.55-1.65 μm, 1.6-1.7 μm, 1.65-1.75 μm, 1.7-1.8 μm, 1.75-1.85 μm, 1.8-1.9 μm, 1.85-1.95 μm, 1.9-2.0 μm, or greater than 2.0 μm.

In certain embodiments, the chitosan, or derivative thereof, is a chitosan salt. The acid addition salt is one which is formed by reaction with a suitable pharmaceutically acceptable acid. The acid may be a mineral acid or an organic acid, such as a carboxylic or dicarboxylic acid, or a dicarboxy-amino acid. Examples of acid addition salts are those formed with acids such as hydrochloric, nitric, sulphuric, acetic, phosphoric, toluenesulphonic, methanesulphonic, benzenesulphonic, lactic, malic, maleic, succinic, lactobionic, fiunaric and isethionic acids, glutamic acid and aspartic acid. In certain embodiments, the chitosan salt can be chitosan hydrochloride, chitosan hydroglutamate, or chitosan hydrolactate.

In preferred embodiments of the invention chitosan or a derivative can be modified by crosslinking. Crosslinking of chitosan can occur chemically, to an agent such as dialdehydes, citric acid, methacrylic acid, lactic acid, or alginate. Chitosan can further be modified by redox gelation carried out with ammonium persulfate and N,N,N′,N′-tetramethylethelynediamine. Chitosan or a derivative thereof can also be formulated with polyol salts, for example glycerol-, sorbitol-, fructose- and glucose-phosphate salts, to form a hydrogel.

The concentration of chitosan in the composition will typically be up to about 5% (w/v), for example, 0.5%, 1%, 2%, 3%, 4% or 5%.

### Antigens

The compositions of the invention can include one or more antigens and chitosan, or a derivative thereof. Thus, the composition may include a number of antigens. The exemplary antigens listed herein can be full length polypeptides, or antigenic fragments thereof. The compositions of the present invention contemplate the use of human immunodeficiency virus antigens, such as, but not limited to gp120, p24, gp41, p17, HIV gag protein, HIV RT protein, HIV Nef protein, HIV pol protein, HIV env protein, HIV Tat protein. The compositions of the present invention contemplate the use of malaria antigens such as, but not limited to, MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa. The compositions of the present invention contemplate the use of hepatitis antigens such as, but not limited to, formalin-inactivated hepatitis virus, HBsAg, HBeAg, HDAgs, HAV proteins and epitopes, HBV proteins and epitopes, HCV proteins and epitopes, HDV proteins and epitopes, and HEV proteins and epitopes. The compositions of the present invention contemplate the use of influenza antigens such as, but not limited to, HA, NA, H5N1, H1N1, H2N2, H3N2, H7N7, H1N2, H9N2, H7N2, H7N3, H10N7, and HPAI A (H5N1). The compositions of the present invention contemplate the use of cancer antigens. Cancer antigens can be tumor associated antigens. The tumor antigens of the patient can be determined in the course of drawing up the diagnosis and treatment plan by standard methods: tumor antigens can easily be detected by immunohistochemistry using antibodies. If the tumor antigens are enzymes, e.g. tyrosinases, they can be detected by enzyme assays. In the case of tumor antigens with a known sequence, the RT-PCR method can be used (Boon, T., et al., 1994; Coulie, P. G., et al., 1994; Weynants, P., et al., 1994). Other methods of detection are assays based on CTLs with specificity for the tumor antigen which is to be detected. These assays have been described, for example, by Herin et al., 1987; Coulie et al., 1993; Cox et al., 1994; Rivoltini et al., 1995; Kawakami et al., 1995; and have been described in WO 94/14459; these references also disclose various tumor antigens and peptide epitopes derived therefrom which are suitable within the scope of the present invention. Examples of suitable tumor antigens are also given in the summarizing articles published recently by Rosenberg, 1996, and Henderson and Finn, 1996. Regarding the tumor antigens which can be used the present invention is not subject to any limitations; some examples of known tumor antigens and peptides derive therefrom which may be used for the purposes of the invention include: the cancer antigen is selected from the group consisting of hTERT, HSPs, Her2/neu, progesterone receptors, androgen receptors, normal or mutated EGFR, CEA, MART-1, MAGE-1, MAGE-3, LAGE-1, LAGE-2, BAGE family antigens, XAGE family antigens, GAGE family antigens, GP-100, MUC-1, MUC-2, point mutated ras oncogene, normal or point mutated p53, CA-125, PSA, PSMA, C-erb/B2, BRCA I, BRCA II, tyrosinase, SCP-1, CT-7, TRP-1, TRP-2, NY-ESO-1, NY-BR-1, NY-BR-1-85, NY-BR-62, NY-BR-85, HOXB7, PDEF, HPV E7, TAG72, TALE, KSA, SART-3, MTAs, WT1, Survivin, Mesothelin, bcr-abl, pax3-fkhr, ews-fli-1, Ku70/80, RCAS1, cytokeratins, stathmin, vimentin, tumor-associated antigen (TAA), tumor specific antigen, whole tumor cells, tissue specific antigen, modified TAAs, splice variants of TAAs, functional epitopes and epitope agonists thereof.

The composition according to the invention can also include chitosan, or derivative thereof, and an antigen to treat any disease wherein an antigen is known or can be readily determined. For example, the depot compositions of the invention can be used to treat diseases including, but not limited to: anthrax, human papilloma virus, tuberculosis, Ebola, West Nile, SARS, Lyme disease, Meningitis, rabies, cholera, yellow fever, encephalitis, CMV, Diptheria, Hib, Measles, Pertussis, Polio, Rubella, TBE, and tetanus.

### Cytokines

The compositions of the invention can include one or more cytokines and chitosan, or a derivative thereof. Additionally, the depot compositions can include one or more antigens, cytokines, and chitosan, or a derivative thereof. In certain embodiments of the invention, the cytokine is administered before administration of the one or more additional vaccines. In other embodiments, the cytokine is administered after administration of the one or more additional vaccines.

Cytokines are extracellular proteins or peptides that mediate cell-cell communication, often with the effect of altering the activation state of cells. Thus, in the invention, cytokines can be used with chitosan, or derivatives thereof, to stimulate an immune response. Exemplary cytokines include interleukins, such as IL-1, IL-2, IL-3, IL-4, IL-5, IL-6, IL-7, IL-8 IL-9, IL-11, IL-11, IL-12, IL-13, IL-17 and IL-18 and IL-23, more particularly, IL-1alpha, IL-1beta, IL-2, IL-7, IL-10, IL-12, IL-15, IL-18, IL-23 and IL-27. In other embodiments, the cytokine is GM-CSF, IFN-alpha, IFN-gamma, IFN-beta, TGF-beta, TNF-alpha, TNF-beta, IL-2, IL-7, IL-12, IL-15, IL-18, IL-23 or IL-27. Chemokines are a subset of cytokines, and accordingly chemokines can be used in the compositions of the invention. There are four main groups of chemokines: C, CC, CXC and CX₃C. Among those groups are specific chemokines that can be used according to the invention, for example: lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1alpha, MIP-1beta, MDC, TECK, TARC, Mig, IP-10, SDF-1alpha/beta, BUNZO/STRC33, I-TAC, BLC/BCA-1, IL-8, BLC, and fractalkine.

### GM-CSF

Certain aspects of the present invention employ methods and preparations that combine chitosan and GM-CSF. GM-CSF (granulocyte macrophage colony stimulating factor) is a hematopoietic growth factor which promotes the proliferation and differentiation of hematopoietic progenitor cells. The cloned gene for GM-CSF has been expressed in bacteria, yeast and mammalian cells. The endogenous protein is a monomeric glycoprotein with a molecular weight of about 22,000 daltons. A recombinant preparation of GM-CSF expressed in bacterial cells is unglycosylated. GM-CSF produced in a yeast expression system is marketed as Leukine® by Immunex Corporation, Seattle, Wash. Leukinel™ is sold in lyophilized form. It is a glycoprotein of 127 amino acids characterized by three primary molecular species having molecular masses of 19,500, 16,800, and 15,500 daltons.

GM-CSF is described in U.S. Pat. No. 5,078,996 to Conlon et al. Analogs of GM-CSF are described in U.S. Pat. Nos. 5,229,496, 5,393,870, and 5,391,485 to Deeley et al. In the certain embodiments, the GM-CSF is a recombinant protein having a molecular weight of between approximately 14,000 and 20,000, and is synthesized in yeast which hyperglycosylates the protein, likely thereby limiting the amount of non-specific absorption observed with the protein. GM-CSF fusion proteins can also be used, e.g., in combination with IL-3 and/or other lymphokines or growth factors.

### Interleukin-12

Certain aspects of the present invention employ methods and preparations that combine chitosan and IL-12 or recombinant IL-12 (rIL-12). Interleukin-12 (IL-12), also known as Natural Killer Cell Stimulating Factor (NKSF), Cytotoxic Lymphocyte Matruation Factor (CLMF) and T-cell Stimulating Factor (TSF) is a heterodimer comprised of two disulfide linked subunits: p35 and p40. Among its many documented properties, IL-12 is a TH1 polarizing pro-inflammatory cytokine that promotes the proliferation and activation of Natural Killer cells and T lymphocytes.

IL-12/NKSF/CLMF/TSF is described by M. Kobayashi et al, J. Exp. Med., 170:827 (1989). The expression and isolation of IL-12 protein in recombinant host cells is described in detail in International Patent Application WO90/05147, published May 17, 1990 incorporated by reference herein. The DNA and amino acid sequences of the 30 kd and 40 kd subunits of the heterodimeric human IL-12 are provided in the above recited international application

### Methods of Treatment

The present invention contemplates methods for producing an immune response in a subject comprising mixing one or more antigens with chitosan, or a derivative thereof, to form a depot, and administering the chitosan antigen depot to the subject, thereby producing an immune response in the subject. In certain embodiments the methods of the invention comprise mixing one or more cytokines with chitosan, or a derivative thereof, to form a depot, for administration to a subject.

The methods of the invention include increasing an immune response in a subject. An immune response encompasses the process whereby inflammatory cells are recruited from the blood to lymphoid as well as non-lymphoid tissues via a multifactorial process that involves distinct adhesive and activation steps. Inflammatory conditions cause the release of chemokines and other factors that, by upregulating and activating adhesion molecules on inflammatory cells, promote adhesion, morphological changes, and extravasation concurrent with chemotaxis through the tissues. An immune response according to the invention can be an adaptive immune response. The immune response encompassed by the methods of the invention can be a cell mediated cell-mediated immune response or a humoral immune response. Cell-mediated immunity is an immune response that does not involve antibodies but rather involves the activation of macrophages and natural killer cells, the production of antigen-specific cytotoxic T-lymphocytes, and the release of various cytokines in response to an antigen. A humoral immune response is that aspect of specific immunity that is mediated by B lymphocytes and is mediated by antibodies.

Various diseases can be treated by the methods of the invention by administration of a depot composition comprising one or more disease antigens and chitosan, or a derivative thereof, to the subject. In this way, identification of a disease antigen will allow treatment of the disease according to the methods of the invention. In certain embodiments, for example, the diseases that can be treated by the methods of the invention are human immunodeficiency virus, cancer, malaria, influenza, and hepatitis.

Accordingly, HIV antigens can be an antigen to an envelope protein. An HIV antigen can be selected from, but not limited to, for example, gp120, p24, gp41, p17, HIV gag protein, HIV RT protein, HIV Nef protein, HIV pol protein, HIV env protein, HIV Tat protein. Cancer antigens can be any tumor associated antigens, for instance hTERT, HSPs, Her2/neu, progesterone receptors, androgen receptors, normal or mutated EGFR, CEA, MART-1, MAGE-1, MAGE-3, LAGE-1, LAGE-2, BAGE family antigens, XAGE family antigens, GAGE family antigens, GP-100, MUC-1, MUC-2, point mutated ras oncogene, normal or point mutated p53, CA-125, PSA, PSMA, C-erb/B2, BRCA I, BRCA II, tyrosinase, SCP-1, CT-7, TRP-1, TRP-2, NY-ESO-1, NY-BR-1, NY-BR-1-85, NY-BR-62, NY-BR-85, HOXB7, PDEF, HPV E7, TAG72, TALE, KSA, SART-3, MTAs, WT1, Survivin, Mesothelin, bcr-abl, pax3-fkhr, ews-fli-1, Ku70/80, RCAS1, cytokeratins, stathmin, vimentin, tumor-associated antigen (TAA), tumor specific antigen, tissue specific antigen, modified TAAs, splice variants of TAAs, functional epitopes and epitope agonists thereof. Malaria antigens can be selected from, for example, MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa. Hepatitis antigens can be any of, but not limited to, formalin-inactivated hepatitis virus, HBsAg, HBeAg, HDAgs, HAV proteins and epitopes, HBV proteins and epitopes, HCV proteins and epitopes, HDV proteins and epitopes, and HEV proteins and epitopes. Influenza antigens can be any of, but not limited to, HA, NA, H5N1, H1N1, H2N2, H3N2, H7N7, H1N2, H9N2, H7N2, H7N3, H10N7, and HPAI A (H5N1).

Particular methods of the invention describe treating or preventing cancer in a subject by administering a depot composition comprising one or more small molecule inhibitors and chitosan, or a derivative thereof, to the subject and thereby treating or preventing cancer. Any small molecule inhibitor is useful according to the methods of the invention. For instance, a double-stranded RNA, siRNA (short interfering RNA), shRNA (short hairpin RNA), or antisense RNA, or a portion thereof, or a mimetic thereof would be useful according to the invention. Specifically, siRNA is a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity. In particular examples, the small molecule inhibitor is selected from the group consisting of: siRNA, shRNA, DNA aptamers, RNA aptamers, and antisense oligonucleotides.

In another embodiment of the method for treating or preventing cancer in a subject, the method comprises administering a depot composition comprising one or antibodies, or fragments thereof, and chitosan, or a derivative thereof, to the subject to treat cancer. The antibody can be any monoclonal or polyclonal antibody that is useful for treating cancer, for instance inhibiting cell proliferation, inhibiting tumor growth, inhibiting cell cycle.

### Nanoparticles and Microparticles

Particular embodiments of the invention describe a method for producing an immune response in a subject comprising mixing particles that are comprised of chitosan or a derivative thereof, containing one or more antigens. The method in certain embodiments can be a method for increasing a cell mediated immune response in a subject that comprises mixing particles comprised of chitosan or a derivative thereof, containing one or more antigens, and delivering the antigen-containing chitosan-based particles to the subject to produce an immune response. The method in other embodiments can be a method for increasing a cell mediated immune response in a subject that comprises mixing particles comprised of chitosan or a derivative thereof, containing one or more cytokines, and delivering the particles to the subject to produce an immune response. The particles are useful for treatment of diseases, for example, but not limited to, HIV, cancer, malaria, influenza and hepatitis, as described above.

The particles can be microparticles or nanoparticles, and range in size from 20 nm to 10 μm. The particles, in certain embodiments, can be multilayered. U.S. Pat. No. 6,649,192, incorporated herein by reference, describes the formation of chitosan nanoparticles. In certain embodiments of the invention the particles can be fabricated via ionotropic gelation, auto-assembly, mechanical disruption of a dried chitosan antigen film, sonication-evaporation or emulsion-diffusion-evaporation. Particles can be further processed by freeze-drying and reconstituted in a buffer of choice.

### Compositions

The present invention contemplates pharmaceutical preparations including depot compositions comprising one or more antigens and chitosan, or a derivative thereof, depot compositions comprising one or more cytokines and chitosan, or a derivative thereof, or depot compositions comprising one or more antigens, one or more cytokines and chitosan, or a derivative thereof. The compositions should be sterile and contain a therapeutically effective amount of the antigens and chitosan, or antigen and cytokine in a unit of weight or volume suitable for administration to a subject.

In certain embodiments, the depot composition is a vaccine depot composition. The depot compositions of the present invention include a chitosan composition as described above and an antigen and/or cytokine. An “antigen” is meant to encompass any antigenic or immunogenic polypeptides including poly-aminoacid materials having epitopes or combinations of epitopes, and immunogen-encoding polynucleotides. In addition, an “antigen” is also meant to encompass any poly-saccharide material useful in generating immune response. As used herein, an antigen, when introduced into a subject, reacts with the immune system molecules of the subject, i.e., is antigenic, and/or induces an immune response in the subject, i.e., is immunogenic. An antigen may be an immunogenic polypeptide. An immunogenic polypeptide will also be antigenic, but an antigenic polypeptide, because of its size or conformation, may not necessarily be immunogenic. Examples of antigenic and immunogenic polypeptides include, but are not limited to, polypeptides from infectious agents such as bacteria, viruses, parasites, or fungi, allergens such as those from pet dander, plants, dust, and other environmental sources, as well as certain self polypeptides, for example, tumor-associated antigens.

In addition, antigenic and immunogenic polypeptides of the present invention can be used to prevent or treat, i.e., cure, ameliorate, or lessen the severity of cancer including, but not limited to, cancers of oral cavity and pharynx (i.e., tongue, mouth, pharynx), digestive system (i.e., esophagus, stomach, small intestine, colon, rectum, anus, anal canal, anorectum, liver, gallbladder, pancreas), respiratory system (i.e., larynx, lung), bones, joints, soft tissues (including heart), skin, melanoma, breast, reproductive organs (i.e., cervix, endometirum, ovary, vulva, vagina, prostate, testis, penis), urinary system (i.e., urinary bladder, kidney, ureter, and other urinary organs), eye, brain, endocrine system (i.e., thyroid and other endocrine), lymphoma (i.e., hodgkin's disease, non-hodgkin's lymphoma), multiple myeloma, leukemia (i.e., acute lymphocytic leukemia, chronic lymphocytic leukemia, acute myeloid leukemia, chronic myeloid leukemia). In addition to the exemplary antigens described above, the instant invention also contemplates the use of the following types of microorganism derived antigens.

Examples of viral antigenic and immunogenic polypeptides include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, e.g., a hepatitis B core or surface antigen, herpesvirus polypeptides, e.g., a herpes simplex virus or varicella zoster virus glycoprotein, immunodeficiency virus polypeptides, e.g., the human immunodeficiency virus envelope or protease, infectious peritonitis virus polypeptides, influenza virus polypeptides, e.g., an influenza A hemagglutinin, neuramimidase, or nucleoprotein, leukemia virus polypeptides, Marburg virus polypeptides, orthomyxovirus polypeptides, papilloma virus polypeptides, parainfluenza virus polypeptides, e.g., the hemagglutinin/neuramimidase, paramyxovirus polypeptides, parvovirus polypeptides, pestivirus polypeptides, picoma virus polypeptides, e.g., a poliovirus capsid polypeptide, pox virus polypeptides, e.g., a vaccinia virus polypeptide, rabies virus polypeptides, e.g., a rabies virus glycoprotein G, reovirus polypeptides, retrovirus polypeptides, and rotavirus polypeptides.

Examples of bacterial antigenic and immunogenic polypeptides include, but are not limited to, Actinomyces polypeptides, Bacillus polypeptides, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides, e.g., H. influenzae type b outer membrane protein, Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, e.g., S. pyogenes M proteins, Treponema polypeptides, and Yersinia polypeptides, e.g., Y. pestis F1 and V. antigens.

Examples of fungal immunogenic and antigenic polypeptides include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite immunogenic and antigenic polypeptides include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, e.g., P. falciparum circumsporozoite (PfCSP), sporozoite surface protein 2 (PfSSP2), carboxyl terminus of liver state antigen 1 (PfLSA1c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of helminth parasite immunogenic and antigenic polypeptides include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyrne polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Additional adjuvants can be included in the compositions of the invention. Examples of additional adjuvants include, but are not limited to basic polyamino acid or a mixture of basic polyamino acids, such as apolyarginine, polylysine, orpolyornithine, or histones, protamines, polyethyleneimines or mixtures thereof. The adjuvant may preferably be CpG motifs, Imiquimod, LPS, MPL, MF59, Ribi Detox™, Alum, QS-21, Freund's complete adjuvant, Freund's incomplete adjuvant, MDP, TDM, ISCOMS, Adjuvant 65, Lipovant, TITERMAX, Montanide ISA720, BCG, Levamisole, squalene, Pluronic, TWEEN, inulin, polyinosinic-polycytidylic acid or any other TLR ligand.

Pharmaceutical compositions of the invention can comprise one or more pH buffering compounds to maintain the pH of the formulation at a predetermined level, such as in the range of about 3.0 to about 9.0. Illustrative examples of such pH buffering compounds include, but are not limited to water, deionized water, PBS, DPBS, HBSS, HEPES, ethanol, methanol, acetic acid, hydrochloric acid, sodium hydroxide solution. The pH buffering compound may be present in any amount suitable to maintain the pH of the formulation at a predetermined level.

Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

### Mode of Administration

According to the present invention, the immunogenic composition of the present invention can be used to produce an immune response in a subject. The method for producing an immune response in a subject includes administering to the subject a composition of the present invention in an amount sufficient to generate an immune response to the composition.

The compositions of the present invention may be administered according to any of various methods known in the art. For example, U.S. Pat. No. 7,105,162 reports on pharmaceutical compositions for immunomodulation.

Specifically, the immunogenic compositions of the present invention may be administered to any tissue of a subject, including, but not limited to, muscle, skin, brain, lung, bladder, liver, spleen, bone marrow, thymus, heart, lymph, blood, bone, cartilage, mucosal tissue, pancreas, kidney, gall bladder, stomach, intestine, testis, ovary, uterus, vaginal tissue, rectum, nervous system, eye, gland, tongue and connective tissue. Preferably, the compositions are administered to skeletal muscle, subcutis, solid tumor or bladder. The immunogenic compositions of the invention may also be administered to a body cavity, including, but not limited to, the lung, mouth, nasal cavity, stomach, peritoneum, intestine, heart chamber, vein, artery, capillary, lymphatic, uterus, vagina, rectum, and ocular cavity.

Preferably, the immunogenic compositions of the present invention are administered by subcutaneous, intradermal, intramuscular, intratumoral injection and intravesical and transdermal delivery. Other suitable routes of administration include, intranasal, inhalation, intratracheal, transmucosal (i.e., across a mucous membrane), intra-cavity (e.g., oral, vaginal, or rectal), intraocular, vaginal, rectal, intraperitoneal, intraintestinal and intravenous (i.v.) administration.

Any mode of administration can be used so long as the administration results in desired immune response. Administration means of the present invention include, but not limited to, needle injection, catheter infusion, biolistic injectors, particle accelerators (i.e., “gene guns” or pneumatic “needleless” injectors—for example, Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet, Medijector, gelfoam sponge depots, other commercially available depot materials (e.g., hydrojels), osmotic pumps (e.g., Alza minipumps), oral or suppositorial solid (tablet or pill) pharmaceutical formulations, topical skin creams, and decanting, use of polynucleotide coated suture (Qin et al., Life Sciences 65, 2193-2203 (1999)) or topical applications during surgery. The preferred modes of administration are intramuscular needle-based injection and intranasal application as an aqueous solution.

Determining an effective amount of an immunogenic composition depends upon a number of factors including, for example, the chemical structure and biological activity of the substance, the age and weight of the subject, and the route of administration. The precise amount, number of doses, and timing of doses can be readily determined by those skilled in the art.

In certain embodiments, the immunogenic composition is administered as a pharmaceutical composition. Such a pharmaceutical composition can be formulated according to known methods, whereby the substance to be delivered is combined with a pharmaceutically acceptable carrier vehicle. Suitable vehicles and their preparation are described, for example, in Remington's Pharmaceutical Sciences, 16.sup.th Edition, A. Osol, ed., Mack Publishing Co., Easton, Pa. (1980), and Remington's Pharmaceutical Sciences, 19.sup.th Edition, A. R. Gennaro, ed., Mack Publishing Co., Easton, Pa. (1995). The pharmaceutical composition can be formulated as an emulsion, gel, solution, suspension, lyophilized form, or any other form known in the art. In addition, the pharmaceutical composition can also contain pharmaceutically acceptable additives including, for example, diluents, binders, stabilizers, and preservatives. Administration of pharmaceutically acceptable salts of the polynucleotide constructs described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like.

For aqueous pharmaceutical compositions used in vivo, use of sterile pyrogen-free water is preferred. Such formulations will contain an effective amount of the immunogenic composition together with a suitable amount of vehicle in order to prepare pharmaceutically acceptable compositions suitable for administration to a vertebrate.

### Kits

The present invention also provides kits for use in delivering a composition to a subject. A kit according to the invention may include a chitosan antigen depot, together with instructions for use. In some embodiments, the kit includes a chitosan cytokine depot, together with instructions for use. The kit can also include a chitosan antigen cytokine depot, together with instructions for use. In preferred kits of the invention, the cytokine is a chemokine. Without being limited in scope, some of the cytokines and chemokines included in the kits within the scope of the invention include IL-1alpha, IL-1beta, IL-2, IL-7, IL-10, IL-12, IL-15, IL-18, IL-23, IL-27, GM-CSF, IFN-alpha, IFN-gamma, IFN-beta, TGF-beta, TNF-alpha, TNF-beta, C, CC, CXC, CX₃C, lymphotactin, MCP-1, MCP-2, MCP-3, MCP-4, MEC, CTACK, 6Ckine, MPIF-1, MIP-5/HCC-2, 1-309, DC-CK1, HCC-1, HCC-4, RANTES, MIP-1 alpha, MIP-1beta, MDC, TECK, TARC, Mig, IP-10, SDF-1 alpha/beta, BUNZO/STRC33, I-TAC, BLC/BCA-1, IL-8, BLC, and fractalkine. The invention further includes kits that increase the efficacy of a vaccine comprising chitosan, and instructions for use.

Each kit includes a container holding an antigen and/or a container holding a cytokine. Furthermore, each kit includes, in the same or in a different container, a composition comprising chitosan, or derivatives thereof. The kit may also include a buffer or solvent. Any of components of the pharmaceutical kits can be provided in a single container or in multiple containers. Any suitable container or containers may be used with pharmaceutical kits. Examples of containers include, but are not limited to, glass containers, plastic containers, or strips of plastic or paper.

Each of the pharmaceutical kits may further comprise an administration means. Means for administration include, but are not limited to syringes and needles, catheters, biolistic injectors, particle accelerators, i.e., “gene guns,” pneumatic “needleless” injectors, gelfoam sponge depots, other commercially available depot materials, e.g., hydrojels, osmotic pumps, and decanting or topical applications during surgery. Each of the pharmaceutical kits may further comprise sutures, e.g., coated with the immunogenic composition (see, for example, Qin et al. 1999 Life Sciences 65:2193-2203).

The kit can further comprise an instruction sheet for administration of the composition to a subject. The components of the pharmaceutical composition are preferably provided as a liquid solution or they may be provided in lyophilized form as a dried powder or a cake. If the composition is provided in lyophilized form, the dried powder or cake may also include any salts, entry enhancing agents, transfection facilitating agents, and additives of the pharmaceutical composition in dried form. Such a kit may further comprise a container with an exact amount of sterile pyrogen-free water, for precise reconstitution of the lyophilized components of the pharmaceutical composition.

The container in which the pharmaceutical composition is packaged prior to use can comprise a hermetically sealed container enclosing an amount of the lyophilized formulation or a solution containing the formulation suitable for a pharmaceutically effective dose thereof, or multiples of an effective dose. The pharmaceutical composition is packaged in a sterile container, and the hermetically sealed container is designed to preserve sterility of the pharmaceutical formulation until use. Optionally, the container can be associated with administration means and/or instruction for use.

This invention is further illustrated by the following examples, which should not be construed as limiting. All documents mentioned herein are incorporated herein by reference.

### EXAMPLES

The following materials and methods were used in the examples described below:

### Methods of the Invention

The results reported herein were obtained using the following Materials and Methods:

### Animals, Antigens and Adjuvants

Female C57BL/6 mice (8-12 weeks old) were obtained from the National Cancer Institute, Frederick Cancer Research Facility (Frederick, Md.). Mice were housed and maintained under pathogen-free conditions in microisolator cages. Animal care was in compliance with recommendations of The Guide for Care and Use of Laboratory Animals (National Research Council). Beta-galactosidase was purchased from Prozyme (San Leandro, Calif.). Ovalbumin (Grade VI) and concanavalin A were purchased from Sigma-Aldrich (St. Louis, Mo.). Chitosan (Protosan G 213) was purchased from NovaMatrix (Drammen, Norway). Incomplete Freund's adjuvant (IFA) was purchased from Rockland (Gilbertsville, Pa.). Aluminum hydroxide (Imject Alum) was purchased from Pierce Biotechnology, Inc. (Rockford, Ill.). Recombinant murine GM-CSF (rGM-CSF) was purchased from Peprotech (Rocky Hill, N.J.). Human Influenza A strain PR/8 purified virus was purchased from Advanced Biotechnologies, Inc. (Columbia, Md.). The influenza virus was inactived via DNA crosslinking during exposure to ultraviolet (UV) light for 10 minutes in a Stratalinker (Stratagene; LaJolla, Calif.). The resulting antigen was subsequently referred to as UV-inactivated influenza.

### Vaccinations

Where beta-galactosidase was injected, vaccinations consisted of a prime and one boost, separated by 1 week, with 100 μg beta-galactosidase. Vaccinations were given as two 50 μl sub cutaneous injections administered bilaterally in the lumbar region. Beta-galactosidase was formulated via simple addition with either PBS or 1.5% chitosan dissolved in PBS. Beta-galactosidase was formulated with aluminum hydroxide or IFA according to the manufacturer's instructions.

For rGM-CSF studies, vaccinations consisted of a prime and a boost, separated by 1 week, with 5 μg UV-inactivated influenza. UV-inactivated influenza was formulated via simple addition with PBS, chitosan alone, rGM-CSF alone or chitosan and rGM-CSF (chitosan/rGM-CSF) together. For the chitosan alone treatment group, 1.5% chitosan (w/v) was dissolved in DPBS prior to mixing with antigen. For the rGM-CSF alone group, antigen was mixed with 20 μg rGM-CSF in saline. Three additional daily s.c. injections of 20 μg rGM-CSF were given at the vaccination site. For the combined group, either 20 μg or 80 μg of rGM-CSF was added to 1.5% chitosan (w/v) dissolved in DPBS (denoted as chitosan/rGM-CSF (20 μg) and chitosan/rGM-CSF (80 μg), respectively). All rGM-CSF study vaccines were administered as a single 100 μl s.c. injection in the lower flank/lumbar region on opposite sides for the prime and boost.

To determine if chitosan enhanced the immune response in subjects administered virally encoded antigens, eight to twelve week old C57BL/6 female mice were vaccinated subcutaneously. Vaccinations consisted of a prime and a boost, separated by 2 weeks, with 1×10⁸ pfu recombinant fowlpox encoding influenza nucleoprotein and a triad of costimulatory molecules (rF-Flu/TRICOM) in either PBS or co-formulated with 1.5% (w/v) chitosan. One week after the booster vaccination, spleens were harvested for lymphoproliferation assays.

To determine if chitosan enhanced the immune response in subjects administered yeast constructs containing antigen, eight to twelve week old C57BL/6 female mice were vaccinated subcutaneously. Vaccinations consisted of a prime and a boost, separated by 1 week, with 1 yeast unit of a recombinant yeast construct containing carcinoembryonic antigen (CEA) in either PBS or co-formulated with 1.5% (w/v) chitosan. Two weeks after the booster vaccination, spleens were harvested for lymphoproliferation assays.

### Intratumoral Chitosan/Cytokine Therapy

For the intratumoral chitosan/cytokine studies, eight to twelve week old C57BL/6 female mice transgenic for human CEA were given 3×10⁵ MC32a (murine colon adenocarcinoma expressing CEA) cells subcutaneously in the flank. In one experiment, 7 and 14 days after implantation, mice were treated intratumorally with 50 μl of PBS, 1.5% chitosan, recombinant Interferon-γ (25 k IU) or recombinant IFN-γ (25 k IU) formulated with 1.5% chitosan. In another experiment, 7, 14 and 21 days after implantation, mice were treated intratumorally with 50 μl of PBS, rIL-12 (1 μg), 1.5% chitosan, 1 μg rIL-12 formulated with 1.5% chitosan (chitosan/rIL-12 (1 μg)) or 5 μg rIL-12 formulated with 1.5% chitosan (chitosan/rIL-12 (5 μg)). Tumor volumes were measured twice per week.

### Splenic CD4⁺ Proliferation Assay

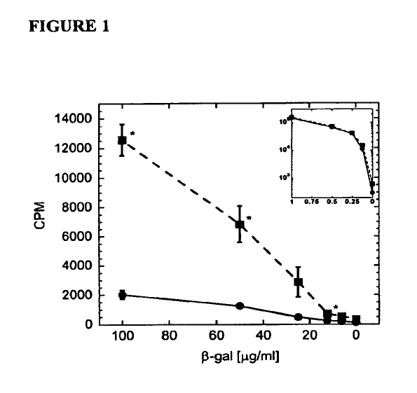

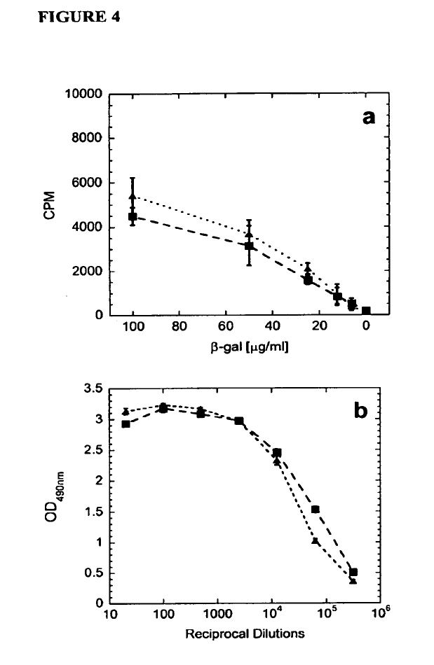

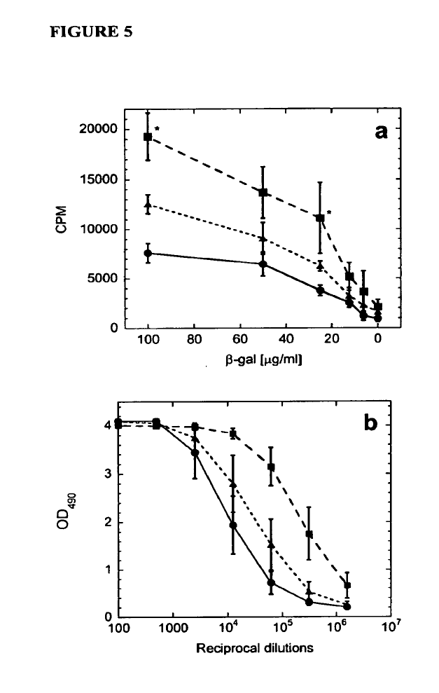

All proliferation assays were initiated 1 week following the booster vaccination and performed as described previously with minor modifications (Kass et al. 2001 Cancer Res 61 (1):206-14). Briefly, harvested spleens were mechanically disrupted with a syringe plunger and passed through a 70 μm nylon mesh strainer (BD Biosciences; Bedford, Mass.). Erythrocytes were lysed with ACK lysing buffer (Cambrex Bio Science; Walkersville, Md.). CD4⁺ splenocytes were isolated via Dynal® CD4 negative isolation kits (Invitrogen; Carlsbad, Calif.) according to the manufacturer's instructions. Splenic CD4⁺ cells from immunized mice at 1×10⁵ (FIG. 4), 1.5×10⁵ (FIG. 1), or (FIG. 5) 2×10⁵ were co-incubated with 5×10⁵ irradiated (20 Gy) naïve syngeneic splenocytes in individual wells of a 96-well plate. Cells were stimulated with 6.25-100 μg/ml beta-galactosidase for 5 days. For positive controls, cells were stimulated with 0.0625-1 μg/ml concanavalin A, a T cell mitogen, for 3 days. For non-specific antigen controls, cells were stimulated with 100 μg/ml ovalbumin for 5 days. In all cases, cells were labeled with 1 μCi/well [³H]-thymidine (Amersham Biosciences; Piscataway, N.J.) for the final 18 h of culture. Following incubation, cultures were harvested onto glass fiber filtermats via a Tomtec Harvester 96 (Hamden, Conn.). Incorporated radioactivity was measured by liquid scintillation counting on a 1450 Betaplate (Perkin-Elmer; Shelton, Conn.). Results from individual mice in triplicate wells were combined to yield a mean±SEM for each immunization group.

For rGM-CSF studies, two hundred-thousand splenic CD4⁺ cells from immunized mice were co-incubated with 5×10⁵ irradiated (20 Gy) naïve syngeneic splenocytes in triplicate wells of a 96-well plate. Cells were stimulated with 0.31-5 μg/ml UV-inactivated influenza for 5 days. For positive controls, cells were stimulated with 0.0625-1 μg/ml concanavalin A, a T cell mitogen, for 3 days. For non-specific antigen controls, cells were stimulated with 50 μg/ml ovalbumin for 5 days. In all cases, cells were labeled with 1 μCi/well [³H]-thymidine (Amersham Biosciences; Piscataway, N.J.) for the final 18 h of culture. Incorporated radioactivity was measured as described above, and results from individual mice in triplicate wells were combined for each immunization group as described above.

### Serum Antibody Responses

Antigen-specific serum antibody responses were measured 1 week following the booster vaccination via ELISA. Briefly, microtiter plates were sensitized overnight at 4° C. with 100 ng/well beta-galactosidase or ovalbumin (as a negative control). Wells were blocked with 5% BSA in PBS for 1 h at 37° C. Wells were then incubated with serum serially diluted (1:20-1:1,526,500). Anti-beta-gal was used as positive control (Promega; Madison, Wis.). Following a 1 h incubation, wells were washed thrice with 1% BSA in PBS and incubated with Horseradish peroxidase-conjugated goat-anti-mouse IgG (Pierce; Rockford, Ill.), IgG₁ or IgG₂ₐ (Southern Biotech; Birmingham, Ala.). Following a 1 h incubation, wells were washed thrice with 1% BSA in PBS and incubated with o-phenylenediamine (Sigma-Aldrich; St. Louis, Mo.) according to the manufacturer's instructions. The reaction was stopped with 3N HCl and the absorbance of each well was read at 490 nm using a Bio-Tek Synergy HT multi-detection microplate reader (Winooski, Vt.).

### Delayed-Type Hypersensitivity

Seven days after the booster vaccination, the baseline thickness of both ears was measured with a spring-loaded dial gauge (Mitutoyo Corp., Tokyo, Japan). Ten minutes prior to antigen challenge, mice were anesthetized with 15 mg/kg xylazine+75 mg/kg ketamine. Ten microliters of PBS or beta-galactosidase (5 mg/ml) were injected into opposite pinnae. Ear thickness was measured in triplicate 24 h after challenge. The thickness of the ear challenged with antigen was divided by the thickness of the ear challenged with PBS to obtain percent increase in ear thickness.

### Flow Cytometry

Inguinal lymph nodes were harvested, via gross dissection, mechanically disrupted with a syringe plunger and passed through a 70 μm nylon mesh strainer (BD Biosciences; Bedford, Mass.). Cells were washed twice with cold PBS. FcγII and FcγIII receptors on lymphocytes were blocked via incubation with 1 μg purified anti-mouse CD16/CD32 (clone: 2.4G2) (BD Biosciences; San Jose, Calif.) per 1×10⁶ cells for 15 min on ice. Cells were stained with fluorescence-labeled antibodies (1 μg/1×10⁶ cells) to the following markers (BD Biosciences; San Jose, Calif.): CD3e (clone: 145-2C11), CD19 (clone: 1D3), CD4 (clone: RM4-5), CD8a (clone: 53-6.7), NK1.1 (clone: PK136), CD25 (clone: PC61), CD11b (clone: M1/70), CD11c (clone: HL3), and Gr-1 (clone: RB6-8C5).

For rGM-CSF studies, mice were given either one 100 μl s.c. injection in the lower flank/lumbar region of PBS or chitosan/rGM-CSF (20 μg) or four daily injections of 20 μg rGM-CSF starting at day 0. Chitosan/rGM-CSF (20 μg) was formulated by adding 20 μg rGM-CSF to 1.5% chitosan (w/v) dissolved in DPBS. The draining inguinal lymph node was harvested, via gross dissection, at the designated times following treatment. Nodes were mechanically disrupted with a syringe plunger and passed through a 70 μm nylon mesh strainer (BD Biosciences; Bedford, Mass.). Cells were washed twice with cold PBS. FcγII and FcγIII receptors on lymphocytes were blocked via incubation with 1 μg purified anti-mouse CD16/CD32 (clone: 2.4G2) (BD Biosciences; San Jose, Calif.) per 1×10⁶ cells for 15 min on ice. Cells were stained with fluorescence-labeled antibodies (1 μg/1×10⁶ cells) to the following markers (BD Biosciences; San Jose, Calif.): CD3e (clone: 145-2C11), CD19 (clone: 1D3), NK1.1 (clone: PK136), CD25 (clone: PC61), CD11b (clone: M1/70), CD11c (clone: HL3), and Gr-1 (clone: RB6-8C5).

For all flow cytometry studies, antibody isotype controls (BD Biosciences; San Jose, Calif.) included: mouse IgG₁ (clone: MOPC-31C), mouse IgG₂ₐ (clone: G155-178), rat IgG₁ (clone: A110-1), rat IgG₂ₐ (clone: R35-95), rat IgG_{2b} (clone: A95-1) and Hamster IgG₁ (clone: A19-3). Following a 45 min incubation on ice, cells were washed twice with cold PBS and read in six colors on a LSR II (BD Biosciences; San Jose, Calif.). Data analyses were performed using BD FACSDiva Software (BD Biosciences; San Jose, Calif.).

### Mixed Lymphocyte Response (MLR)

Draining inguinal lymph nodes were harvested following rGM-CSF treatment as before (section 2.3). Cells were counted, irradiated (20 Gy) and serially diluted (from 5×10⁵ to 1.56×10⁴ cells/well) in triplicate in a 96-well plate. T cells from Balb/c mice were obtained following B220 depletion of splenocytes using Dynal® B220 isolation kits (Invitrogen; Carlsbad, Calif.) according to the manufacturer's instructions. Five hundred-thousand Balb/c T cells were co-incubated with the irradiated lymphocytes for 4 days. Cells were labeled with 1 μCi/well [³H]-thymidine (Amersham Biosciences; Piscataway, N.J.) for the final 18 h of culture. Following incubation, cultures were harvested onto glass fiber filtermats via a Tomtec Harvester 96 (Hamden, Conn.). Incorporated radioactivity was measured by liquid scintillation counting on a 1450 Betaplate (Perkin-Elmer; Shelton, Conn.). Results from individual mice in triplicate wells were combined to yield a mean±SEM for each treatment group.

### Non-Invasive Fluorescence Imaging of Antigen Depots

Non-invasive animal imaging was carried out in the Mouse Imaging Facility (MIF), a division of the NIH MRI Research Facility (NMRF). Fluorescence and photographic images of anesthetized mice that were given a single s.c. injection of Alexa Fluor 660-labeled beta-galactosidase formulated in either PBS or 1.5% chitosan or were given Alexa Fluor 660-labeled rGM-CSF formulated in PBS, 1% or 2% chitosan (w/v) were acquired over a 2-week period with an IVIS100 Imaging System (Xenogen; Alameda, Calif.). Anesthesia was induced in a chamber with 4-5% isoflurane delivered by a gas mixture of oxygen, nitrogen and medical air. Once mice were unconscious and unresponsive to toe pinch, anesthesia was maintained with 1-2% isoflurane administered via nosecone. Following each imaging session, mice were allowed to recover in the MIF/NMRF on a circulating warm water pad until they could breathe unassisted and walk. Prior to the initial imaging session, the lumbar regions of mice were shaved with electric shears. Residual hair was removed with a depilatory cream. Approximately 60 μg of beta-galactosidase, labeled with an Alexa Fluor 660 protein labeling kit (Invitrogen; Carlsbad, Calif.) or approximately 20 μg of rGM-CSF, labeled with an Alexa Fluor 660 protein labeling kit (Invitrogen; Carlsbad, Calif.), were injected s.c. in a total volume of 50 μl. The fluorescence intensity of the injection site was used as a surrogate for beta-galactosidase or rGM-CSF concentration. The fluorescence intensity of a region of interest drawn around the injection site was calculated at each time point with Living Image® software (Xenogen; Alameda, Calif.). Background/autofluorescence from non-injected control mice was subtracted. Fluorescence data for each mouse were normalized by the initial measurement, which was taken immediately after injection, for that mouse.

### Pentamer Staining

Pro5® MHC Class I pentamers were purchased from ProImmune (Oxford, UK). Splenocytes directly from mice or after a 7 day in vitro stimulation with peptide were stained with a pentamer specific for an H-2D^{b} epitope for influenza A/PR/8 nucleoprotein (Flu NP₃₆₆₋₃₇₄; ASNENTETM; SEQ ID NO: 1) or a control pentamer specific for an H-2D^{b} epitope for lymphocytic choriomeningitis virus nucleoprotein (LCMV NP₃₉₆₋₄₀₄; FGPQNGQFI; SEQ ID NO: 2) according to the manufacturer's instructions. Cells were read and analyzed as before on an LSR II.

### Cytotoxic T Cell Lysis (CTL) Assay

For rGM-CSF experiments, one week after the booster vaccination, spleens from vaccinated mice were harvested as before. Approximately 25×10⁶ unfractionated splenocytes from each vaccine group were cultured in an upright T-25 flask containing 10 ng/ml Flu NP₃₆₆₋₃₇₄ peptide (ASNENTETM; SEQ ID NO: 1) (CPC Scientific; San Jose, Calif.). After one week, lymphocytes were collected on a histopaque (Sigma-Aldrich; St. Louis, Mo.) density gradient and quantified. Target EL-4 cells (4×10⁶) were radiolabeled in RPMI 1640 with 50 μCi in ¹¹¹In-labeled oxine (GE Healthcare; Silver Spring, Md.) for 30 minutes at 37° C. Target cells were washed twice in complete media and pulsed with 1 μg/ml Flu NP₃₆₆₋₃₇₄ or HIV gag₃₉₀₋₃₉₈ (control) peptide for 30 minutes at 37° C. Five thousand target cells/well were co-incubated with 5 to 500×10³ lymphocytesin triplicate wells in a 96-well plate for 18 h at 37° C. The amount of ¹¹¹In released was measured using a gamma counter (Cobra II; Packard Instruments, Downers Grove, Ill.). The percentage of specific lysis was calculated as follows:

$$\% specific lysis = \frac{Experimental cpm - spontaneous cpm}{Maximal cpm - spontaneous cpm} \times 100$$

The reported % lysis was Flu NP₃₆₆₋₃₇₄ specific lysis subtracted from HIV gag₃₉₀₋₃₉₈ specific lysis.

### Histopathology

Similar to the vaccinations described above, mice (n=9) were given bilateral s.c. injections of 50 μl of 1.5% chitosan in the lumbar region. Mice were sacrificed 2, 7 or 14 days after the injection. The skin/subcutis containing the injection site was removed, embedded in paraffin, sectioned and stained with hematoxylin and eosin to document inflammation and chitosan regression. Slides were blinded and read by a board certified pathologist.

### Statistical Analysis

Statistical analyses of differences between means of antigen-specific splenic CD4⁺ proliferation, antibody titer, CTL, lymph node cell numbers and lymphocyte percentages from flow cytometry experiments were performed using Student's two-tailed t test assuming unequal variances (JMP Software; Cary, N.C.). Differences in means were accepted as significant if P was less than 0.05.

### Example 1

### Chitosan Enhanced Both Humoral and Cell-Mediated Vaccine Responses

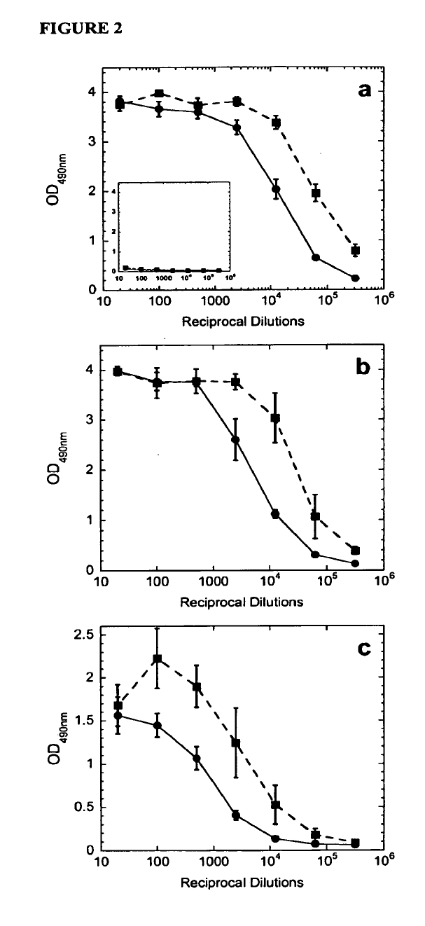

C57BL/6 mice were vaccinated subcutaneously with a model antigen, beta-galactosidase, in either PBS or chitosan solution. Proliferation of CD4⁺ splenocytes from mice receiving the vaccine in chitosan is significantly greater (P<0.05) than that of CD4⁺ splenocytes from mice receiving the vaccine in PBS when re-exposed to the vaccine antigen, as shown in FIG. 1. Chitosan also increased serum IgG titers to beta-galactosidase (FIG. 2a). Antibody titers in mice administered beta-galactosidase in chitosan were increased 5.3 fold, as linearly approximated at an optical density of 1.0. Similarly, chitosan enhanced antigen-specific IgG₁ and IgG₂ₐ titers 5.9- and 8.0-fold respectively, implying a mixed T_{H}1/T_{H}2 response (FIGS. 2b-c). All increases in antibody titers were statistically significant (P<0.001).

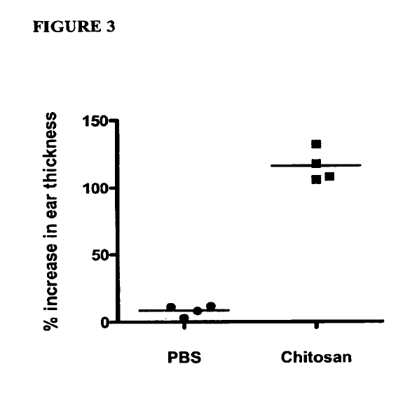

Delayed-type hypersensitivity responses were measured as an in vivo assay of cell-mediated immune function. One week after the booster vaccination, mice were challenged with 50 μg of beta-galactosidase in the pinnae. Opposite pinnae were injected with PBS to control for non-specific inflammation. Twenty-four hours after challenge, mice originally vaccinated with beta-galactosidase in PBS had, on average, less than a 10% increase in ear thickness. However, mice originally vaccinated with beta-galactosidase formulated with chitosan had a substantial 116% increase in ear thickness indicating a robust cell-mediated immune response, as seen in FIG. 3.

### Example 2

### Chitosan was Equipotent to IFA and Superior to Aluminum Hydroxide

After it was shown that chitosan had vaccine enhancing properties, the next objective was to compare chitosan with the commonly used adjuvants, IFA and aluminum hydroxide. Antigen-specific CD4⁺ proliferative and serum antibody responses were similar in mice vaccinated with beta-galactosidase in either chitosan solution or IFA, as shown in FIG. 4a-b. Antigen-specific CD4⁺ proliferative responses were significantly greater (P<0.05) in mice vaccinated with beta-galactosidase in a chitosan solution rather than aluminum hydroxide (FIG. 5a). Chitosan also enhanced antigen-specific antibody titers 6.6-fold over aluminum hydroxide at optical density of 1.0, as shown in FIG. 5b.

### Example 3

### Chitosan Expanded Local Lymph Nodes

During the aforementioned studies, mice were dissected to note any gross pathological changes that resulted from the subcutaneous (s.c.) injection of chitosan. A significant increase in the size of the lymph nodes draining the s.c. chitosan injections was observed. Mice were otherwise healthy at the time of sacrifice. To characterize the leukocyte expansion, inguinal lymph nodes were resected, disrupted, counted and stained for phenotypic analysis via six-color flow cytometry. The number of leukocytes in inguinal lymph nodes from mice injected with chitosan increased by more than 67% from 4.9×10⁶ leukocytes per node at day 0 to 8.2×10⁶ leukocytes per node at day 14, shown in Table 1, below. Table 1 shows the results of experimentation wherein chitosan, without antigen, was injected subcutaneously at Day 0. Spleens and lymph nodes were harvested at Days 0, 2, 7, 14 and 21. The results in Table 1 show that chitosan increased the number of lymphocytes per ILN by 67% (Day 14). Chitosan modestly increased the number of NK1.1⁺ cells in the lymph node and spleen as well as the number of CD11b⁺ cells in the lymph nodes. All other compartments were not significantly altered by chitosan. Data are represented as the mean (SD) of 5 mice. * indicates P<0.05 compared to Day 0.

TABLE 1
The effect of chitosan on the total number and percent of lymphocyte
subsets in the spleen and inguinal lymph nodes (ILN).

| | lymphocyte | | | | | | | |
|---|---|---|---|---|---|---|---|---|
| | number | CD3⁺ | CD19⁺ | CD8⁺ | NK 1.1⁺ | Gr-1⁺ | CD11c⁺ | CD11b⁺ |
| | Spleen | | | | | | | |
| Day 0 | 114.8 (14.4) | 31.6 (2.4) | 60.4 (2.5) | 12.2 (1.4) | 4.6 (0.3) | 5.7 (0.3) | 3.4 (0.3) | 4.4 (0.6) |
| Day 2 | 118.4 (13.4) | 28.5 (1.9) | 62.2 (2.4) | 12.6 (1.0) | 5.6 (0.6)* | 5.5 (0.2) | 3.7 (0.2) | 5.0 (0.4) |
| Day 7 | 117.6 (22.6) | 31.3 (2.7) | 59.5 (2.5) | 12.3 (0.8) | 5.7 (0.6)* | 5.7 (0.3) | 3.5 (0.2) | 4.9 (0.3) |
| Day 14 | 116.9 (8.2) | 28.7 (1.4) | 63.4 (2.0) | 10.6 (0.6) | 4.6 (0.3) | 5.5 (0.5) | 3.2 (0.4) | 4.0 (0.4) |
| Day 21 | 112.5 (19.3) | 31.5 (2.6) | 56.2 (3.6) | 12.5 (1.1) | 5.1 (0.5)* | 5.9 (0.5) | 3.2 (0.2) | 4.3 (0.5) |
| | ILN | | | | | | | |
| Day 0 | 4.9 (0.4) | 67.6 (6.0) | 24.8 (5.2) | 28.0 (2.8) | 1.5 (0.1) | 7.4 (1.0) | 1.2 (0.4) | 1.5 (0.2) |
| Day 2 | 6.2 (0.9)* | 62.9 (3.2) | 30.7 (5.0) | 25.8 (2.0) | 2.8 (0.4)* | 7.8 (1.4) | 1.8 (0.6) | 2.7 (0.8)* |
| Day 7 | 7.4 (1.1)* | 65.3 (1.3) | 30.0 (1.5) | 27.9 (0.9) | 3.6 (0.4)* | 7.8 (7.2) | 1.9 (0.7) | 2.8 (0.7)* |
| Day 14 | 8.2 (0.8)* | 66.6 (2.2) | 30.2 (2.3) | 26.6 (1.1) | 1.8 (0.2) | 8.6 (0.8) | 1.1 (0.2) | 1.8 (0.3) |
| Day 21 | 6.6 (0.6)* | 66.1 (3.1) | 30.9 (3.0) | 26.0 (1.2) | 1.8 (0.2) | 8.2 (0.5) | 1.0 (0.2) | 1.5 (0.2) |

### Example 4

### Chitosan Retained Antigen at the Injection Site

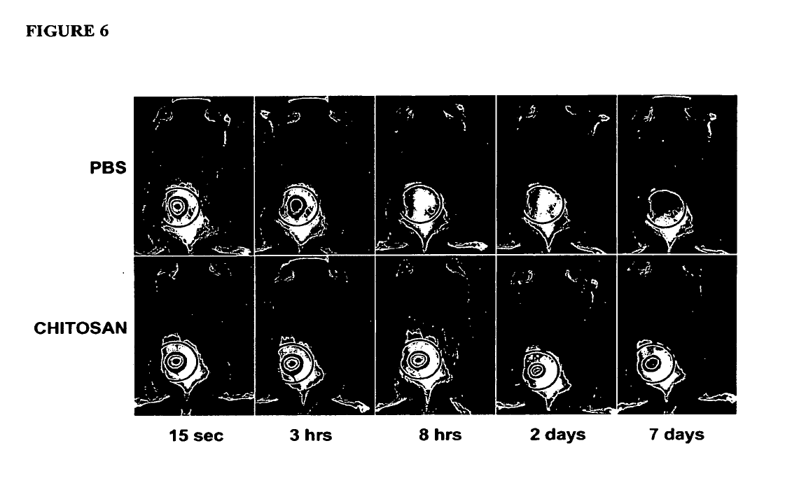

Another possible mechanism by which vaccine was enhanced included establishment and maintenance of an antigen depot at the injection site. Dissemination of macromolecules from an injection site can be hindered greatly by highly viscous solutions (Wang et al. 2003 Mol Cancer Ther 2(11):1233-42). The use of 1.5% chitosan solution, which, according to the manufacturer, was approximately two orders of magnitude more viscous than water, was expected to result in a depot of antigen at the injection site. To verify this hypothesis, beta-galactosidase was labeled with Alexa Fluor 660 prior to injection in order to track the spatiotemporal distribution of antigen when administered in PBS versus chitosan solution. Mice receiving a single subcutaneous (s.c.) injection with Alexa Fluor 660-labeled beta-galactosidase were imaged over the course of 2 weeks. The results are shown in FIG. 6.

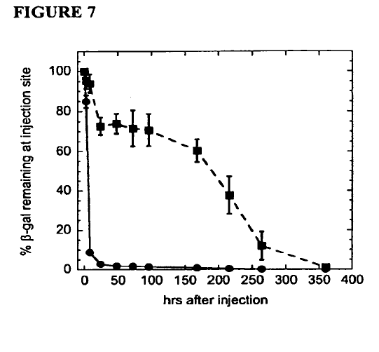

Fluorescence intensity was used as a surrogate for β-galactosidase concentration. Analysis of the injection site revealed that within 24 h, less than 3% of the antigen delivered in PBS remained at the injection site (FIG. 7). This contrasted with greater than 60% of antigen delivered in chitosan having remained present 7 days after injection.

### Example 5

### Chitosan was Highly Biodegradable

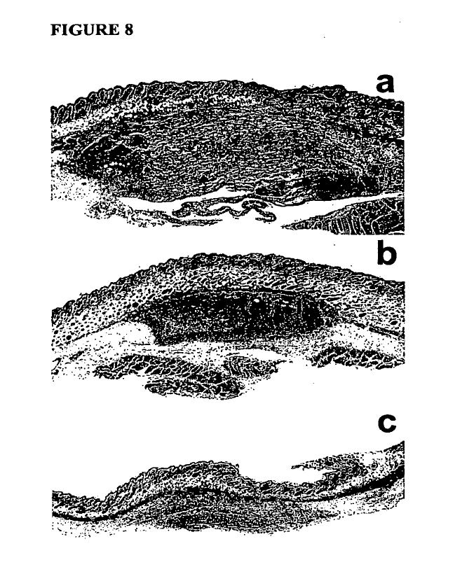

In order to document pathological changes in the subcutis, chitosan solution alone was injected as in the vaccination studies. Tissues surrounding the subcutaneous injection site of chitosan were removed 2, 7 and 14 days after injection and stained with hematoxylin and eosin. Histopathological analysis revealed that chitosan was infiltrated and degraded, mainly by macrophages and neutrophils, in 2-3 weeks (FIG. 8a-c). This rate of degradation coincided with the dissipation of antigen from the injection site (FIG. 7).

### Example 6

### Chitosan Retained Recombinant GM-CSF (rGM-CSF) at an Injection Site

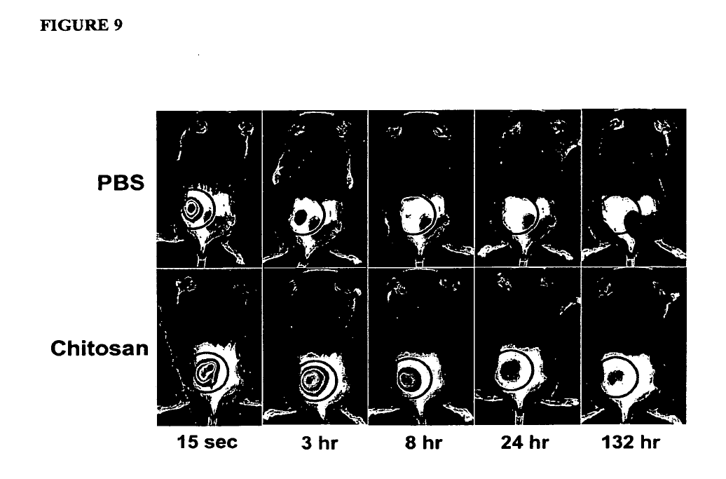

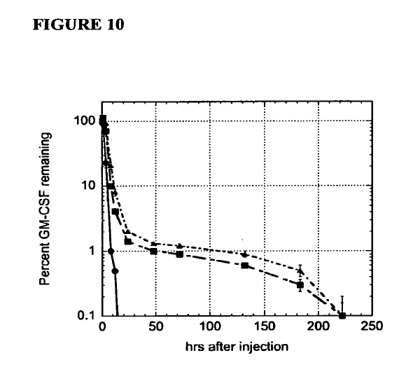

As identified above, chitosan solution, due primarily to its high viscosity, was found to maintain a depot of recombinant protein antigen effectively (also refer to Zaharoff et al. 2007 Vaccine 25(11):2085-94). To show that a cytokine depot could similarly be maintained through use of chitosan, similar fluorescence imaging studies were performed with a cytokine, recombinant GM-CSF (rGM-CSF). Mice received a single s.c. injection of Alexa Fluor 660-labeled rGM-CSF and were imaged over the course of 2 weeks in order to track the spatiotemporal distribution of cytokine delivered in either PBS or chitosan solution (FIG. 9). Fluorescence intensity was used as a surrogate for rGM-CSF concentration. Analysis of the injection site revealed that when delivered in PBS, rGM-CSF was undetectable in 12 to 24 hrs. In contrast, rGM-CSF was measurable for 9 to 10 days when administered in a chitosan solution (FIG. 10). There was no significant difference between 1% and 2% chitosan solution in rGM-CSF retention time. Integration of the area under the curve (AUC) in FIG. 10 showed that total rGM-CSF exposure was increased approximately 3-fold when rGM-CSF was formulated in a chitosan solution.

### Example 7

### Chitosan/rGM-CSF Expanded Local Lymph Nodes

Previous studies demonstrated that rGM-CSF given as four daily s.c. injections of 20 μg transiently expanded local draining lymph nodes in mice (Kass et al. 2000 Cytokine 12(7):960-71). This expansion was accompanied by a significant increase in the number of antigen presenting cells in the local lymph nodes. In this study, the cellular expansion of the draining (inguinal) lymph node was quantified and cells were phenotyped as a function of time (a) to verify the in vivo bioactivity of rGM-CSF when formulated with chitosan, and (b) to understand the temporal relationship between rGM-CSF residence and lymph node expansion. Mice were given either one s.c. injection of either PBS (control), chitosan/rGM-CSF (20 μg) or chitosan/rGM-CSF (80 μg) or four daily injections of 20 μg rGM-CSF starting at day 0. Recombinant GM-CSF alone induced the expected 2- to 3-fold cellular expansion of the draining lymph node at day 7 (Table 2). This expansion was markedly reduced by day 14 and returned to control levels by day 35. The same total dose of rGM-CSF formulated in chitosan, i.e. chitosan/rGM-CSF (80 μg), induced a 3.4-fold cellular expansion of the draining lymph node at day 7 and a 2.1-fold expansion at day 14 before returning to control levels by day 35. Chitosan/rGM-CSF (20 μg) generated the maximal response, at one-fourth the rGM-CSF dose, with a 4.6-fold cellular expansion of the draining lymph node at day 7 and a 3.1-fold expansion at day 14 before returning to control levels by day 35 (Table 2).

TABLE 2
The effect of rGM-CSF on the total number and percent of lymphocyte subsets in the draining inguinal lymph node.

| | Control | | | rGM-CSF (20 μg × 4) | | |
|---|---|---|---|---|---|---|
| Days | 7 | 14 | 35 | 7 | 14 | 35 |
| total cells per LN (10⁶) | 5.0 (0.7) | 4.7 (1.5) | 4.0 (0.5) | 14.0 (3.0)* | 8.2 (1.7)* | 4.6 (0.4) |
| percent I-A^{b+} | 27.9 (3.5) | 36.8 (5.0) | 31.6 (5.5) | 41.1 (5.2)* | 37.3 (4.3) | 36.6 (5.0) |
| # of I-A^{b+} cells (10⁶) | 1.4 (0.3) | 1.7 (0.4) | 1.3 (0.2) | 5.7 (1.3)* | 3.1 (1.0)* | 1.7 (0.3) |
| percent CD11c⁺I-A^{b+} | 1.5 (0.1) | 1.5 (0.2) | 1.9 (0.3) | 2.2 (0.4)* | 1.7 (0.3) | 2.0 (0.6) |
| # of CD11c⁺I-A^{b+} cells (10⁴) | 7.8 (1.7) | 6.9 (1.9) | 7.8 (1.5) | 30.0 (8.7)* | 14.3 (5.7)* | 9.2 (2.7) |
| percent CD11c⁺I-A^{b+}CD80⁺ | 0.5 (0.1) | 0.4 (0.1) | 0.6 (0.1) | 0.6 (0.2) | 0.5 (0.1) | 0.6 (0.1) |
| # of CD11c⁺I-A^{b+}CD80⁺ cells (10⁴) | 2.6 (0.6) | 1.8 (0.3) | 2.4 (0.6) | 8.8 (3.8)* | 4.2 (1.6)* | 2.7 (0.7) |
| | Chitosan/rGM-CSF (20 μg) | | | Chitosan/rGM-CSF (80 μg) | | |
| Days | 7 | 14 | 35 | 7 | 14 | 35 |
| total cells per LN (10⁶) | 23.2 (6.9)** | 14.4 (5.2)* | 4.6 (1.3) | 17.2 (2.6)* | 9.8 (3.3)* | 6.3 (2.5) |
| percent I-A^{b+} | 44.6 (2.1)* | 44.9 (4.8)** | 34.7 (6.4) | 44.8 (7.7)* | 31.3 (3.5) | 35.0 (5.6) |
| # of I-A^{b+} cells (10⁶) | 10.4 (3.5)** | 6.5 (2.8)** | 1.7 (0.7) | 7.8 (2.3)* | 3.0 (0.7)* | 2.2 (0.8) |
| percent CD11c⁺I-A^{b+} | 2.3 (0.3)* | 1.8 (0.2)* | 2.1 (0.3) | 2.3 (0.7) | 1.4 (0.2) | 2.2 (0.2) |
| # of CD11c⁺I-A^{b+} cells (10⁴) | 53.4 (15.2)** | 26.5 (12.1)* | 9.7 (3.5) | 39.7 (14.4)* | 13.2 (4.4)* | 13.8 (5.5) |
| percent CD11c⁺I-A^{b+}CD80⁺ | 0.8 (0.2)* | 0.6 (0.1)* | 0.6 (0.1) | 0.9 (0.3) | 0.4 (0.1) | 0.6 (0.1) |
| # of CD11c⁺I-A^{b+}CD80⁺ cells (10⁴) | 17.4 (3.5)** | 8.0 (3.0)** | 2.7 (0.8) | 14.9 (6.5)* | 3.8 (1.4)* | 3.8 (1.8) |

*Statistically significant (P < 0.05) from control at the respective timepoint
**[bold] Statistically significant (P < 0.05) from rGM-CSF(4 × 20 μg) and control at the respective timepoint

Because of the documented ability of GM-CSF to recruit dendritic and other antigen presenting cells to local lymph nodes (Kass et al. 2001 Cancer Research 61(1):206-14), these subsets were specifically quantified. MHC Class II expression was used to quantify broadly the number of antigen presenting cells (dendritic cells, B cells and monocyte/macrophages) in the draining lymph node. Recombinant GM-CSF alone induced significant increases in percentage, at day 7, and number, at days 7 and 14, of cells expressing the MHC II molecule, I-A^{b} (Table 1). The percentages and numbers of I-A^{b+} cells were increased further by formulating rGM-CSF in chitosan solution. In particular, chitosan/rGM-CSF (20 μg) and chitosan/rGM-CSF (80 μg) induced 7.4- and 5.6-fold increases, respectively, in the number of I-A^{b+} cells in the draining lymph node 7 days after administration. Chitosan/rGM-CSF (20 μg)-mediated increases in the numbers of I-A^{b+} cells in the draining lymph node were significantly greater (P<0.05) than rGM-CSF alone treatment at days 7 and 14. All percentages and numbers of I-A^{b+} cells returned to control levels by day 35.

Dendritic cells, denoted as CD11c⁺I-A^{b+}, were specifically quantified as they are considered the most potent antigen presenting cells. Similar to the I-A^{b+} cells, rGM-CSF alone induced significant increases in percentage, at day 7, and number, at days 7 and 14, of dendritic cells (Table 1). The chitosan/rGM-CSF (20 μg) treatment group maintained significantly higher percentages and numbers of dendritic cells up to day 14. The number of dendritic cells induced by chitosan/rGM-CSF (20 μg) were significantly greater (P<0.05) than those induced by rGM-CSF alone treatment. Chitosan/rGM-CSF (80 μg) treatment also generated significant increases in dendritic cells although not to the level of chitosan/rGM-CSF (20 μg).

Because of the overall expansion of the lymph nodes, the differences between groups in the numbers of dendritic cells per node were magnified. For example, the total numbers of CD11c⁺I-A^{b+} cells were increased 3.8-fold with rGM-CSF alone (P<0.05 vs. control), 6.8-fold with chitosan/rGM-CSF (20 μg) (P<0.05 vs. rGM-CSF alone) and 5.1-fold with chitosan/rGM-CSF (80 μg) (P<0.05 vs. control) at day 7. One week later, numbers of CD11c⁺I-A^{b+} cells in all treatment groups remained significantly greater than control. Interestingly, the 3.8-fold increase in number of CD11c⁺I-A^{b+} cells in the chitosan/rGM-CSF (20 μg) group at day 14 was equal to the maximum 3.8-fold increase in the rGM-CSF group occurring at day 7. This indicated that chitosan not only increased but also sustained the adjuvant properties of rGM-CSF. All percentages and numbers of CD11c⁺I-A^{b+} cells returned to control levels by day 35. Differences in mature dendritic cells, denoted as CD11c⁺CD80⁺, between the treatment groups followed similar trends. For instance, at day 7, numbers of CD11c⁺CD80⁺ cells were increased 3.4-, 6.7- and 5.7-fold for rGM-CSF alone, chitosan/rGM-CSF (20 μg) and chitosan/rGM-CSF (80 μg) treatments, respectively. In total, chitosan/rGM-CSF (20 μg) administration induced the maximum lymph node expansion and the maximum increases in antigen presenting cells in the draining lymph node. There were no significant changes in the percentages of Gr-1⁺, CD11b⁺, Gr-1⁺CD11b⁺, NK1.1⁺ or CD4^{÷}CD25⁺ cells in the draining lymph node with either treatment. There was an approximate 10% decrease in the percentage of CD3⁺ cells and a corresponding 10% increase in percentage of CD19⁺ cells in all treatment groups at day 7. Percentages of both subsets returned to control levels by day 14.

### Example 8

### Chitosan/rGM-CSF Enriched Antigen Presentation in Local Lymph Nodes

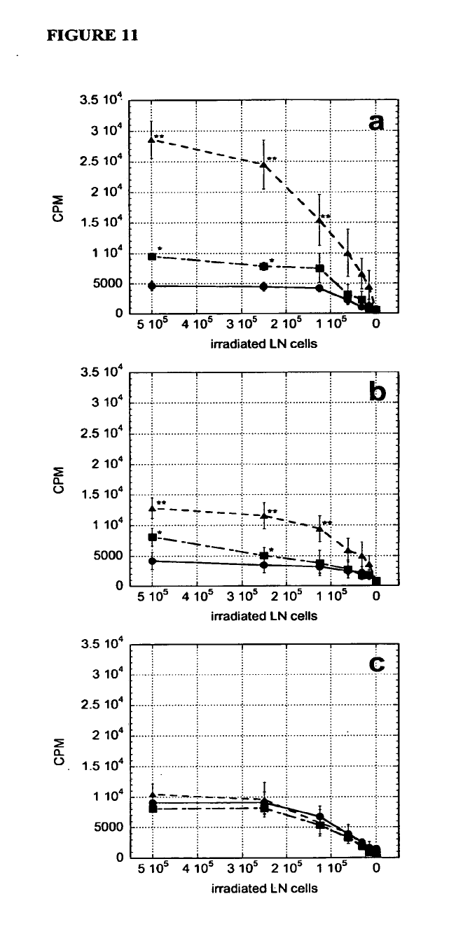

Recombinant GM-CSF administration was previously shown to enhance the antigen presenting ability of lymph node cells (Kass et al. 2000 Cytokine 12(7):960-71). The proliferation of allogeneic T cells co-incubated with irradiated lymph node cells from treated mice was used as a measure of antigen presenting ability. Administration of rGM-CSF alone led to a doubling in antigen presenting ability at day 7 (FIG. 11a-c). However, this enhancement was lost by day 14. On the other hand, chitosan/rGM-CSF mediated about a 5.9-fold increase in antigen presenting ability that remained elevated at day 14 before returning to control levels at day 35.

### Example 9

### Chitosan/rGM-CSF Improved Vaccine Response More than Either Agent Alone

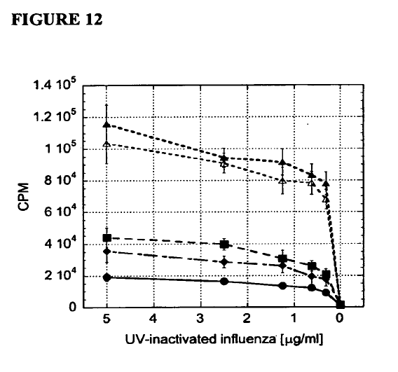

After it was demonstrated that chitosan solution could maintain a depot of functional rGM-CSF that improved antigen presentation, the chitosan/rGM-CSF formulation was tested for its ability to improve a vaccine response. Mice were vaccinated subcutaneously with UV-inactivated influenza formulated with PBS, chitosan solution, rGM-CSF alone, chitosan/rGM-CSF (20 μg) or chitosan/rGM-CSF (80 μg) as described above. Mice that received the antigen in either chitosan solution or rGM-CSF alone exhibited significantly greater antigen-specific proliferation of CD4⁺ splenocytes (P<0.05) than mice that received the antigen with no adjuvant (PBS; FIG. 12). However, administration of antigen in either chitosan/rGM-CSF (20 μg) or chitosan/rGM-CSF (80 μg) resulted in profoundly increased immune responses over either adjuvant alone, demonstrating at least an additive, if not synergistic, enhancement in such response. Similar results were observed using β-galactosidase as the vaccine antigen (data not shown). It was noteworthy that the lower dose rGM-CSF adjuvant, chitosan/rGM-CSF (20 μg), generated an equally robust, if not enhanced, immune response, as compared to the higher dose rGM-CSF adjuvant, chitosan/GM-CSF (80 μg).

### Example 10

### Chitosan Solution Allowed for Lower rGM-CSF Dosage

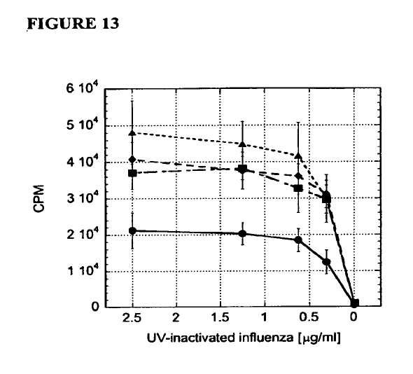

A subsequent vaccination experiment was performed to determine if rGM-CSF doses could be further reduced when formulated with chitosan solution. Vaccines consisted of 0, 5, 10 or 20 μg rGM-CSF formulated with antigen (UV-inactivated influenza) in chitosan solution. Chitosan/rGM-CSF (20 μg) generated the maximum proliferation of antigen-specific CD4⁺ splenocytes; however, comparable responses were observed with chitosan/rGM-CSF (10 μg) and chitosan/rGM-CSF (5 μg) (FIG. 13).

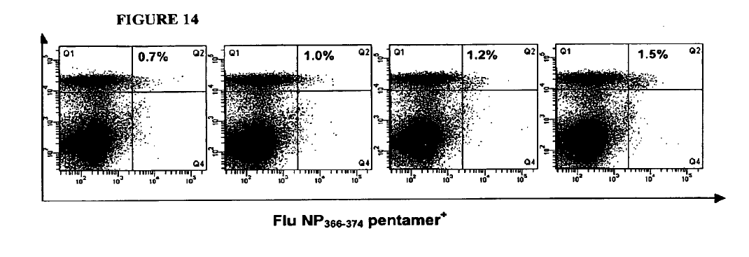

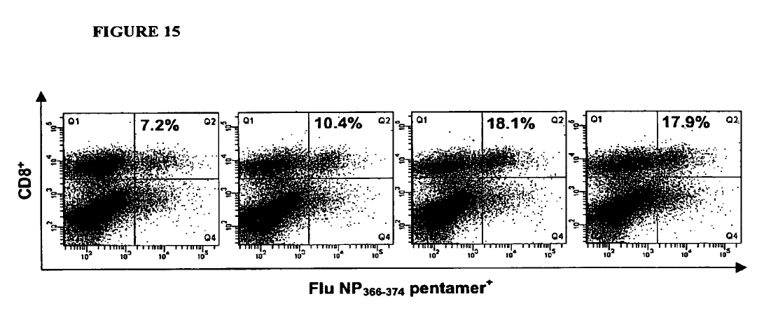

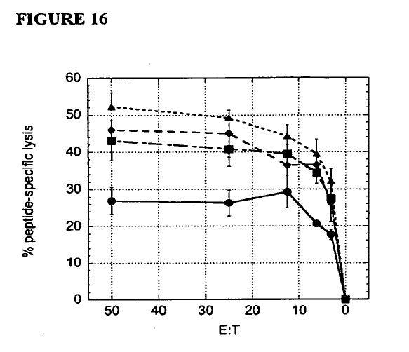

Pentamer staining of fresh splenocytes revealed incremental increases in the percent of CD8⁺ cells specific for Flu NP₃₆₆₋₃₇₄ peptide (ASNENTETM; SEQ ID NO: 1), from 0.7% to 1.5%, as the dose of rGM-CSF in chitosan increased from 0 μg to 20 μg (FIG. 14). When splenocytes were cultured for one week with exogenous peptide, pentamer staining increased substantially and differences between vaccination groups were more pronounced (FIG. 15). Vaccination using either chitosan/rGM-CSF (10 μg) or chitosan/rGM-CSF (20 μg) as adjuvant resulted in nearly one out of every five CD8^{÷} cells staining positive for the Flu NP₃₆₆₋₃₇₄ pentamer (FIG. 16).

To determine if the peptide-specific CD8⁺ cells were cytolytic, cells from the in vitro stimulation studies were co-incubated with peptide-pulsed targets in an overnight CTL assay. Peptide-specific lysis was maximal in the chitosan/rGM-CSF (20 μg) group, which achieved greater than 50% lysis at an E:T ratio of 50:1. As seen above, comparable results were observed for lower doses of rGM-CSF in chitosan, i.e., chitosan/rGM-CSF (5 μg) and chitosan/rGM-CSF (10 μg).

### Example 11

### Chitosan Nanoparticles can be Manufactured in a Range of Sizes

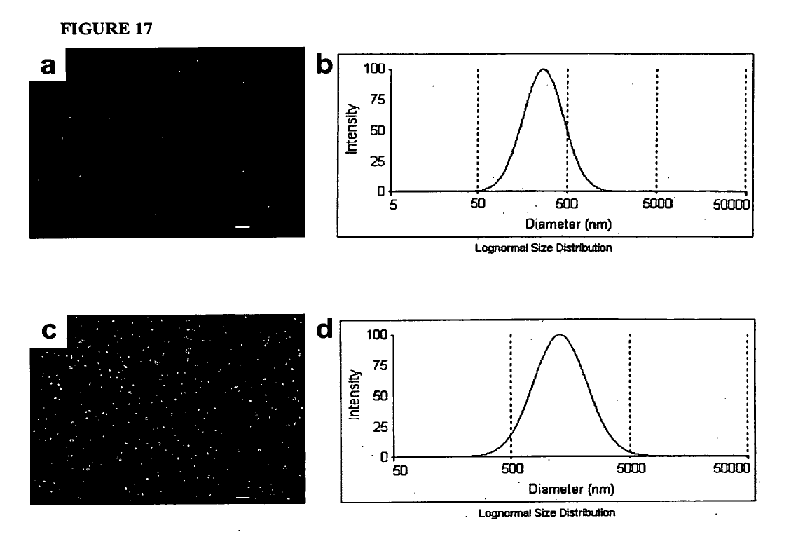

Clinical uses of chitosan can include use of chitosan nanoparticles having a range of sizes. Exemplary effective diameters of batches of manufactured chitosan nanoparticles include 262.1 nm and 1283.2 nm, though a distribution of sizes was observed in such preparations. A lognormal size distribution of chitosan particles with an effective diameter of 262.1 nm was demonstrated to have an encapsulation efficacy of 61% for FITC-BSA (FIG. 17a-b), while a lognormal size distribution of chitosan particles with an effective diameter of 1283.2 nm was demonstrated to have an encapsulation efficacy of 72% for FITC-BSA (FIG. 17c-d).

### Example 12

### Phagocytosis of FITC-BSA/Chitosan Nanoparticles by JAWS II Cells

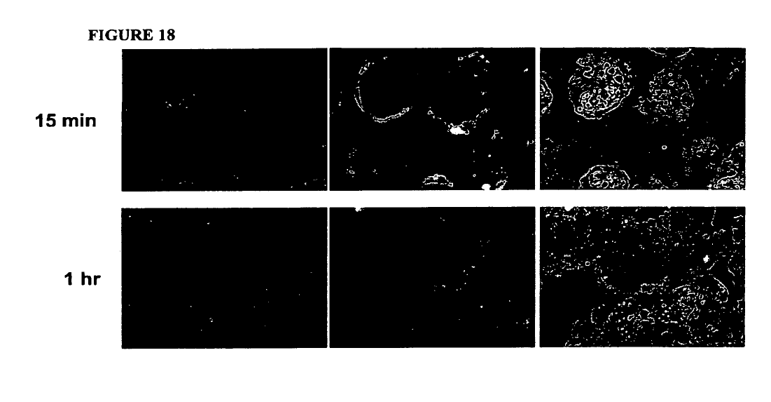

Cellular uptake via phagocytosis of FITC-BSA/chitosan was examined in JAWS II mouse immature dendritic cells. These murine dendritic cells were observed to phagocytose chitosan nanoparticles in less than one hour, indicating successful delivery of chitosan encapsulated payloads to such cells (FIG. 18).

### Example 13

### Uptake of FITC-BSA Encapsulated in Chitosan Nanoparticles into JAWS II Cells In Vitro

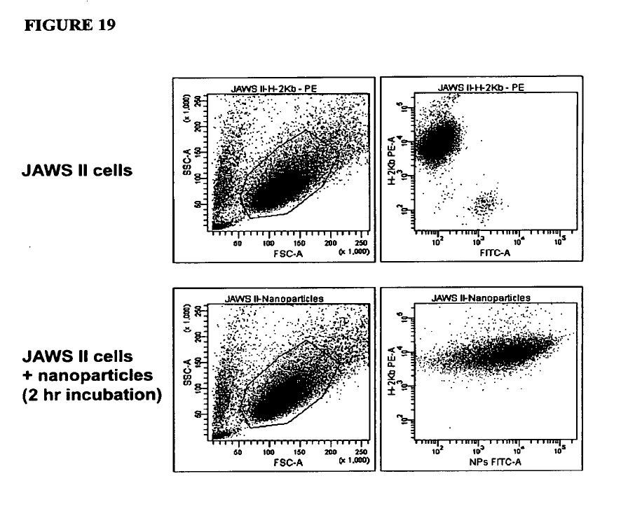

Flow cytometry analysis was used to assess the uptake of FITC-BSA encapsulated in chitosan nanoparticles into JAWS cells in culture. Following a 2 hour incubation of JAWS II cells with FITC-BSA chitosan nanoparticles, it was observed that these murine dendritic cells (JAWS II) took up FITC-BSA chitosan nanoparticles, resulting in a shift in fluorescence, as compared to a control population of JAWS II cells that did not receive FITC-BSA chitosan nanoparticle treatment (FIG. 19).

### Example 14

### Uptake of FITC-BSA Encapsulated in Chitosan Nanoparticles into Bone-Marrow-Derived Dendritic Cells In Vitro

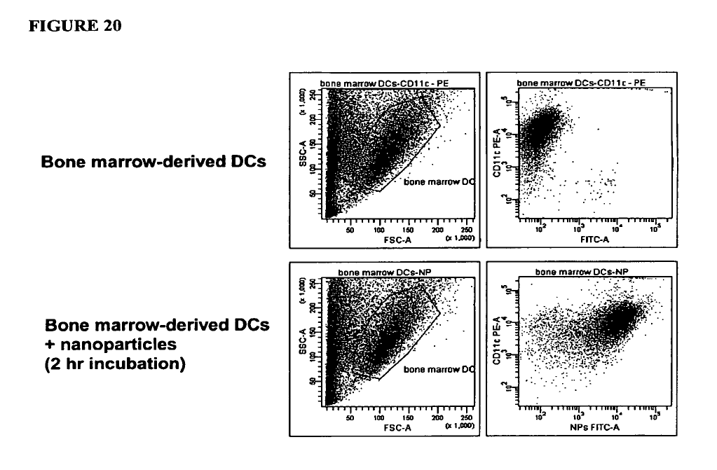

Flow cytometry analysis was used to assess the uptake of FITC-BSA encapsulated in chitosan nanoparticles into bone-marrow-derived dendritic cells in culture. Following a 2 hour incubation of bone-marrow-derived dendritic cells with FITC-BSA chitosan nanoparticles, it was observed that these treated bone-marrow-derived dendritic cells took up FITC-BSA chitosan nanoparticles, resulting in a shift in fluorescence, as compared to a control population of bone-marrow-derived dendritic cells that did not receive FITC-BSA chitosan nanoparticle treatment (FIG. 20). These results confirmed that the uptake of FITC-BSA chitosan nanoparticles was not cell line-dependent, as the fluorescence of bone marrow-derived dendritic cells was shifted in a manner similar to that observed for JAWS II cells in Example 13.

### Example 15

### In Vivo Lymphocyte Uptake of FITC-BSA Encapsulated in Chitosan Nanoparticles

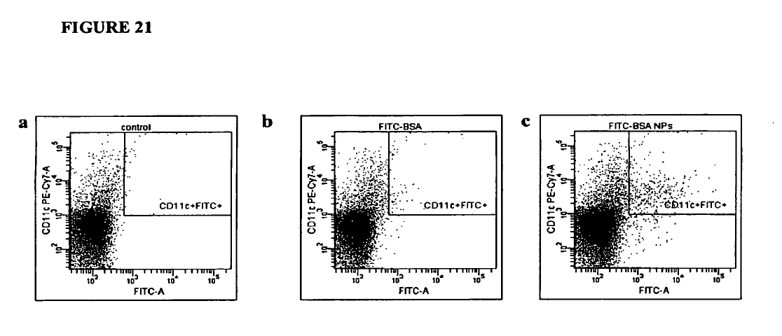

To assess in vivo uptake of FITC-BSA encapsulated in chitosan nanoparticles, mice were administered a single s.c. injection of PBS, FITC-BSA or FITC-BSA encapsulated in chitosan nanoparticles. Twenty-four hours later, the draining inguinal lymph nodes were removed from such mice, and lymphocytes were characterized via flow cytometry for presence of FITC signal and CD11c. It was observed that 1.1% of all lymph node cells (lymphocytes) were double positive for CD11c (CD11c⁺) and FITC, indicating that a FITC-BSA uptake was dramatically enhanced in chitosan nanoparticle preparations relative to PBS and FITC-BSA treatments that did not employ chitosan nanoparticles (CD11c⁺Fitc⁺ levels of 0.1% and 0.2%, respectively; FIGS. 21a-c).

### Example 16

### β-gal/Chitosan Nanoparticle Preparations for Vaccination Enhanced CD4+ Immune Responses in Mice

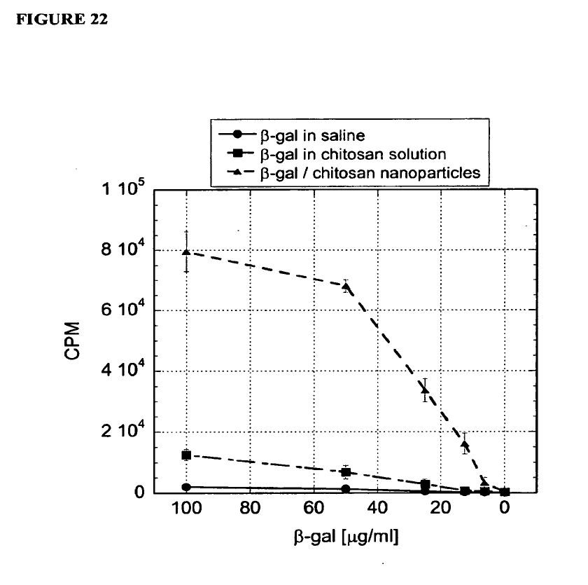

To assess whether use of chitosan nanoparticle preparations in vaccines could enhance the CD4⁺ immune response in vivo, C57BL/6 mice were administered vaccine regimens containing β-gal antigen in saline solution, β-gal antigen in chitosan solution, and β-gal antigen encapsulated in chitosan nanoparticles. Such vaccine regimens involved administration of an initial priming dose at day 0, followed by administration of a booster at day 7, and euthanization and assessment of the CD4⁺ immune response to such treatments at day 14. The antigen-specific CD4⁺ immune response was observed to be especially dramatic for chitosan nanoparticle vaccination regimens (FIG. 22). Mice vaccinated with β-gal antigen in chitosan nanoparticles generated much stronger immune responses than mice vaccinated with either antigen alone or antigen in chitosan solution. These results, when combined with those of the above Examples, indicated that such an effect is likely to be generalizable to any antigen, cytokine, or other polypeptide for in vivo delivery (e.g., to immune cells).

### Example 17

### Determining the Range of Chitosan as a Vaccine Adjuvant

The above examples demonstrated that chitosan was effective for enhancing the adaptive immune response to a model protein antigen, β-galactosidase, as well as a model cytokine, rGM-CSF, following a subcutaneous administration. It is therefore expected that chitosan will enhance the adaptive immune responses to other protein antigens as well. For example, chitosan will enhance the adaptive immune response to other forms of vaccines such as whole tumor cells, virally encoded antigens, yeast constructs containing antigen and peptides. Similarly, as shown for rGM-CSF, chitosan is expected to improve the efficacy of other (recombinant) cytokines. Because chitosan has the ability to form a macromolecular depot and expand local lymph nodes, chitosan is a universal vaccine adjuvant that will enhance the immune response to any type of vaccination. As such, chitosan will also improve the efficacy of short lived, systemically toxic cytokines by forming local depots, consistent with those results observed for rGM-CSF administration with chitosan presented herein.

Further examination of these phenomena will be performed, and include studies that measure the adaptive immune response to a range of additional antigens in vivo. Examples of such antigens include: whole tumor cell vaccine (such as apoptotic and necrotic tumor cells), infectious virally encoded antigens (Fowlpox-based vaccines), inactivated virus (such as Flu), peptides, carbohydrates, subunit vaccines, lipid antigens, and additional glycoprotein antigens.

### Example 18

### Chitosan Enhances Vaccination with Yeast Constructs Containing Antigen but is Detrimental to Vaccination with Recombinant Fowlpox-Encoding Antigen

In previous experiments, we demonstrated for the first time that a viscous chitosan solution maintained a subcutaneous depot and enhanced the adaptive immune response to a model protein antigen following subcutaneous vaccination (Zaharoff et al., Vaccine 25, 2085-94, 2007). In subsequent experiments, we demonstrated that chitosan could 1) improve a vaccine response to inactivated influenza virus and 2) enhance the immunoadjuvant properties of recombinant GM-CSF (Zaharoff et al., submitted). Although, chitosan could improve vaccinations with two diverse antigen types, it was unknown if chitosan would enhance other diverse antigen types such as virus-encoded antigen and yeast constructs containing antigen. The objective of this experiment was to determine if a chitosan could improve a subcutaneous vaccine response, via co-formulation, with either recombinant fowlpox-encoding antigen or yeast constructs encoding antigen.

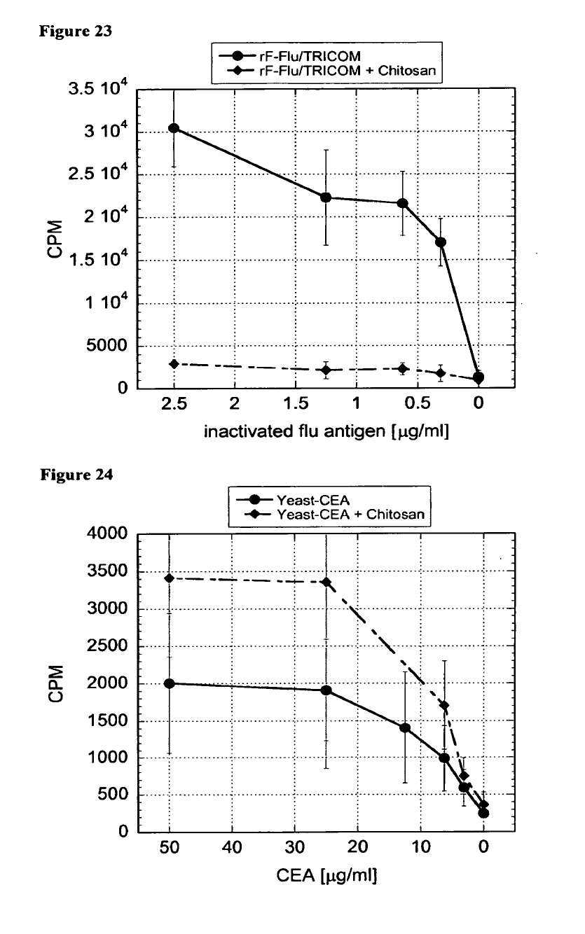

Chitosan eliminated the adaptive immune response to the fowlpox encoded antigen (FIG. 23) but nearly doubled the adaptive immune response to the yeast construct containing antigen (FIG. 24).

### Example 19

### Additional Chitosan-Based Cytokine Depots

Cytokines are very powerful immune response mediators, but are usually short lived and can be systemically toxic at large doses. A depot of cytokine at a vaccination site has been shown to significantly enhance an adaptive immune response. However, there are no clinically approved cytokine depots. As shown above for rGM-CSF, chitosan was not only able to maintain a depot of protein antigen, but was also able to maintain a depot of cytokine. Additional cytokines that are anticipated to form chitosan-based cytokine depots in a manner parallel to rGM-CSF are IL-2, IL-7, IL-12, IL-15, IFN-gamma, and IFN-alpha.

### Example 20

### IL-12-Chitosan Formulations for Intratumoral Administration

The objective of this experiment was to determine if a novel formulation of chitosan and rIL-12 could control a clinically relevant non-immunogenic tumor when injected intratumorally. Interleukin-12 (IL-12) is a strong Th1 polarizing cytokine that drives the activation of NK and CD8⁺ T cells. Recombinant IL-12 (rIL-12) has been used in numerous preclinical immunotherapies and as a vaccine adjuvant. In the clinic, the use of rIL-12 has been limited by its systemic toxicity. Never before has rIL-12 been formulated in a polymer solution for the purposes of (a) controlling its systemic dissemination and (b) prolonging its residence following a parenteral injection and thereby potentiating its immunomodulatory properties. Never before has a polymer-based depot of rIL-12 been injected intratumorally for the purposes of controlling and/or eradicating tumors.

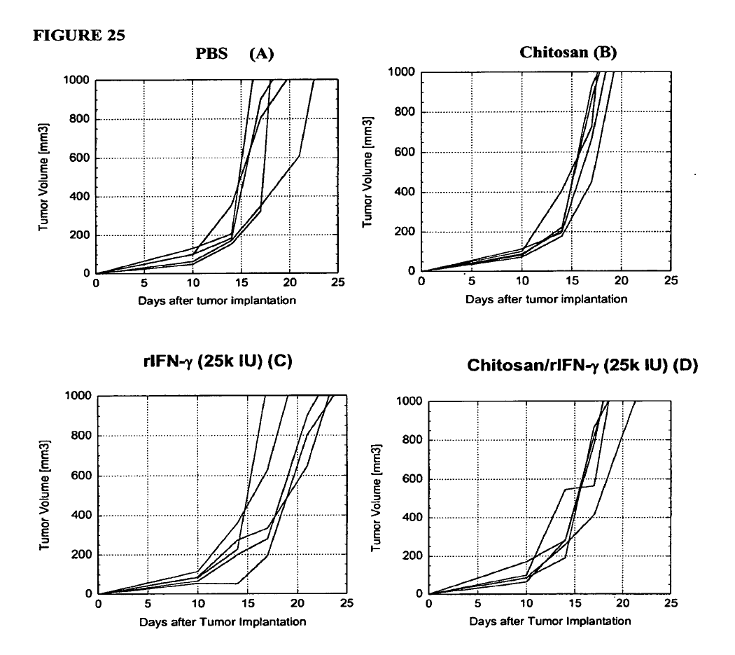

In previous experiments, we demonstrated for the first time that a viscous chitosan solution maintained a subcutaneous depot of functional recombinant cytokine which enhanced a vaccine response. In a subsequent experiment, intratumoral injections of chitosan formulated with an inflammatory Th1 polarizing cytokine, interferon-gamma (IFN-γ) did not delay the growth of transplanted tumors in mice (FIG. 25). Therefore, it was surprising that rIL-12 when formulated with chitosan solution would eradicate tumors when administered intratumorally.

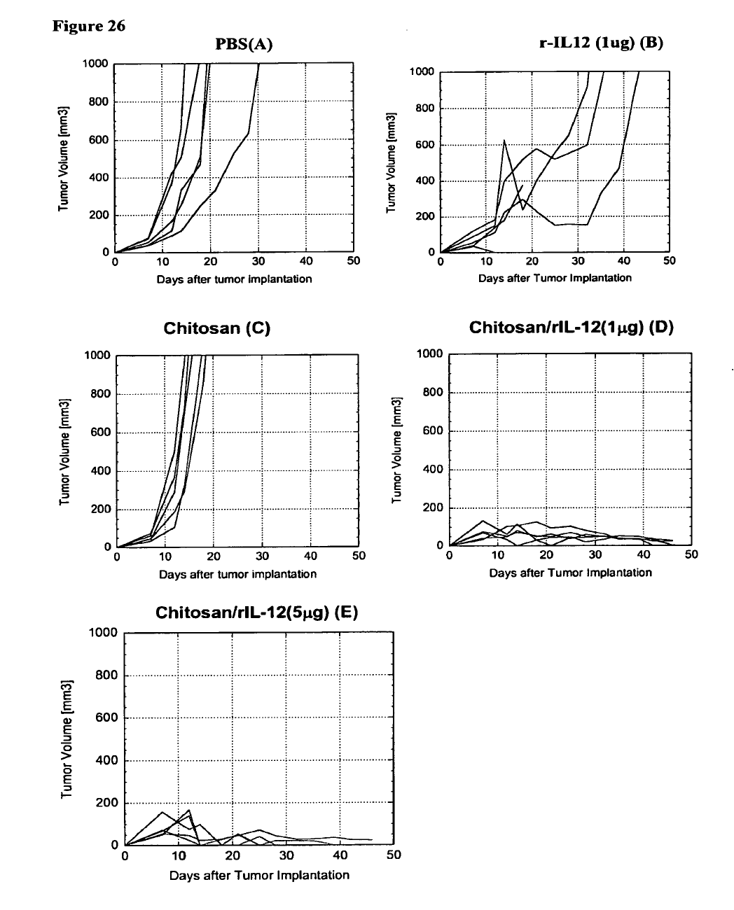

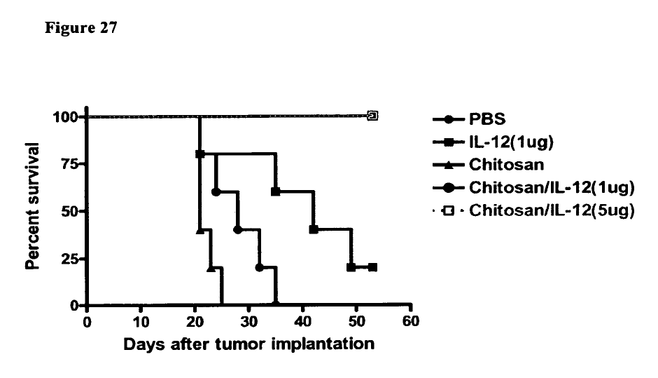

Treatment with chitosan alone did not control tumor progression versus PBS (control) treated mice (FIG. 26). Recombinant IL-12 alone delayed tumor growth modestly and completely eradicated one tumor. One mouse from the rIL-12 group died prematurely with a tumor less than 400 mm³. This may have been due to systemic toxicity of IL-12. Chitosan/rIL-12(1 μg) and chitosan/rIL-12 (5 μg) treatments eradicated tumors in 10 out of 10 mice (FIG. 27). In sum, intratumoral administration of chitosan/rIL-12 demonstrated powerful antitumor effects in an aggressive subcutaneous tumor model. This novel immunotherapy has significant clinical implications in the control of numerous solid tumors that can be injected intratumorally.

### Example 21

### Optimization of Chitosan

Chitosan is a diverse class of polymers whose adjuvant properties can be modified in three ways. First, controlling the conditions in which chitosan is formulated with a biological agent by manipulating chitosan concentration, buffer type, buffer concentration and pH. The formulation environment will be controlled by manipulating the concentration of chitosan, the buffer or solvent used, and the pH. Second, the chitosan molecule itself will be controlled by manipulating molecular weight, degree of deacetylation or derivitizing certain functional groups. For example, derivatization can be selected from: Carboxymethyl-, Hydroxyethyl-, Dihydroxypropyl-, Acetyl-, Phosporylated-, Sulphonated-, N-acetyl-, N-proprionyl-, N-butyryl-, N-pentanoyl- and N-hexanoyl-, glycol-chitosans. Chitosan salts can be used, for example chitosan hydrochloride, chitosan hydroglutamate, chitosan hydrolactate. Third, chitosan will be cross-linked and rendered thermosensitive. Crosslinking can be carried out with dialdehydes, citric acid, methacrylic acid, lactic acid or alginate. Further, external chemical modification will be used to create thermosensitive gels via redox gelation with ammonium persulfate and N-tetramethylethelynediamine or via formulation with polyol salts such as glycerol-, sorbitol-, fructose- and glucose-phosphate salts.

### Example 22

### Chitosan Microparticles and Nanoparticles

In general, it has been demonstrated that antigens in particulate form are more immunogenic. This is due to higher local concentrations of antigen as well as a preference of antigen presenting cells of particulate matter. As demonstrated above, antigens delivered in chitosan nanoparticles elicit a stronger immune response than when mixed in a solution, even though much less antigen (<10%) is delivered. Thus, nanoparticles or microparticles will be used for delivery. To do so, additional optimal particle formulation parameters will be determined. Parameters to consider include the polyanion: sodium phosphate, sodium sulfate, the chitosan to anion ratio, the pH, and the mixing conditions. The encapsulation efficiency will be measured, and additional vaccination experiments will be performed. The data presented above (refer, e.g., to Example 16) have demonstrated that chitosan nanoparticles outperform chitosan solution in enhancing an antigen-specific CD4⁺ responses with a lower dose (<10%) of antigen.

### Example 23

### Mechanistic Studies

Subcutaneous injections of chitosan have been shown to enhance adaptive immune responses by 1) forming an antigen depot and 2) expanding local lymph nodes. To further understand lymph node expansion vaccination studies will be repeated while blocking certain subpopulations to determine their involvement in the enhanced immune response. Also, it is possible that chitosan is a Toll-like receptor (TLR) agonist. Numerous TLRs have been shown to have immune enhancing properties through a wide array of mechanisms. To this end, subpopulation blocking studies will be performed in vivo. Subpopulations of cells that can be blocked include: NK, macrophages, CD4+, CD8+. TLR agonist studies will be performed to determine in vitro cell phenotype characterization and in vivo cell phenotype characterization.

### Example 24

### Varying the Route of Administration

Subcutaneous injections are the most popular route of administration in animal vaccination studies. However, other routes are likely to generate an adaptive immune response as well. Intradermal vaccination will exploit antigen presenting cells residing in the skin. Intramuscular vaccinations are easy to administer in humans and are likely to provide an additional depot function. In tumor bearing mice, intratumoral injections have shown promise in the delivery of cancer vaccines. Enhanced delivery of cancer vaccines can be performed using chitosan nanoparticle preparations. Such nanoparticle cancer vaccine preparations represent advanced cancer therapies due to (1) preference of antigen presenting cells (dendritic cells) for particulate antigen; (2) the flexibility of such nanoparticle preparations (e.g., the ability to incorporate additional agents as knowledge of immunology in the art evolves); (3) the versatility of such nanoparticles (e.g., chitosan nanoparticles can elicit both T_{H}1 and T_{H}2 responses); and (4) the ability of such chitosan nanoparticle preparations to be protected from proteolytic enzymes. For at least the aforementioned reasons, chitosan nanoparticle preparations represent a promising, versatile, translatable platform for the delivery of cancer vaccines, especially in view of the fact that chitosan is a biodegradable, natural biopolymer that forms nanoparticles in mild, aqueous conditions. Multiple sites/routes of vaccination will be evaluated for the compositions described herein. For example, the adaptive immune response will be measured following vaccination via multiple routes, including: subcutaneous, intradermal, intramuscular, intratumoral, intravesical and mixed routes.

### Example 25

### Combination with Other Adjuvants

Because chitosan has been demonstrated to be effective in maintaining protein depots, it is expected that chitosan can maintain a depot of other macromolecules which have been shown to have adjuvant ability. Such a depot will be effective in controlling adjuvant distribution and allowing lower doses of potentially toxic adjuvants to be used. Thus, the adaptive immune responses will be measured when incorporating additional adjuvants, such as, for example, CpG, Imiquimod, Lipopolysaccharide (LPS), and Monophosphoryl Lipid A (MPL), MF59, RIBI DETOX™, Alum, QS-21, Freund's complete adjuvant, Freund's incomplete adjuvant, MDP, TDM, ISCOMS, polyinosinic-polycytidylic acid or any other TLR ligand.

### Example 26

### Pre- or Post-Vaccination Enrichment of Vaccination Site

As shown for rGM-CSF, chitosan alone and chitosan-based cytokine depots can be used to recruit immune cells to enrich a site prior to vaccination or sustain immunological activity post vaccination. Thus, in further studies, chitosan will be delivered with or without rGM-CSF or other cytokines to enrich a vaccination site. Cytokines that can be delivered include, for example: GM-CSF, IL-2, IL-7, IL-12, IL-15, IL-18, IL-23, IL-27, IFN-gamma, IFN-alpha, Lymphotactin, RANTES, and MIP-1alpha (including recombinant forms of all such cytokines).

### Example 27

### The Toxicity of Chitosan

Over 100 adjuvants have been tested preclinically only to fail approval in humans due to toxicity concerns. Chitosan has been shown to be highly biodegradable and there are very few signs of distress in any animal injected with chitosan. Thus, a full toxicology screen after multiple chitosan administrations will be performed. The full toxicology screen will include necropsy, hematology, and blood chemistry analysis.

### Example 28

### Multi-Layered Chitosan Particles

To determine if cancer vaccines delivered via multi-layer antigen- and cytokine-loaded micro- or nanoparticles produce more robust immune and anti-tumor responses than the same vaccines delivered in simple aqueous solutions, the following experiments will be preformed. Multi-layer nanoparticles will be formulated to release antigen or vaccine-enhancing cytokines at appropriate intervals. Exemplary agents to be released include, for example, cytokines such as GM-CSF, IL-2, IL-7, IL-12, IL-15, IL-18, IL-23, IL-27, IFN-gamma, and IFN-alpha. The method will involve imaging the in vivo processing of subcutaneous cancer vaccinations at injection sites and draining lymph nodes using in vivo fiber optic confocal fluorescence. The method will further entail measuring the adaptive immune response in mice vaccinated with a self antigen or dominant peptide and two cytokines in multi-layer chitosan nanoparticles.

### Example 29

### Chitosan Depot to Eliminate Immune Suppressive Factors

Tumors release numerous immunosuppressive factors, such as TGF-beta, VEGF, IL-5, IL-6, IL-10 and IL-16, which facilitate their escape from immunosurveillance. Numerous strategies have been employed to attempt to eliminate or diminish these factors including the use of monoclonal antibodies, polyclonal antibodies, antibody fragments, siRNA, DNA aptamers and RNA aptamers. Chitosan depots containing one or more of the molecules listed above will be formulated for intratumoral administration in order to achieve long term elimination of immunosuppressive factors.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for producing an immune response in a subject, the method comprising the steps of:
i) mixing one or more antigens with chitosan, or a derivative thereof, to form a depot; and
ii) administering the chitosan antigen depot to the subject;
thereby producing an immune response in the subject.

2. A method for increasing a cell mediated immune response in a subject, the method comprising the steps of:
i) mixing one or more antigens with chitosan, or a derivative thereof; and
ii) administering the chitosan antigen depot to the subject;
thereby increasing the cell mediated immune response in the subject.

3. A method for increasing a humoral immune response in a subject, the method comprising the steps of:
i) mixing one or more antigens with chitosan, or a derivative thereof; and
ii) administering the chitosan antigen depot to the subject;
thereby increasing the humoral immune response in the subject.

4. The method of claim 1, wherein the chitosan antigen mixture is administered by one or more routes selected from the group consisting of subcutaneous, intradermal, intramuscular, intratumoral injection and intravesical and transdermal administration.

5. A method for treating or preventing human immunodeficiency virus in a subject, the method comprising the step of:
i) administering a depot composition comprising one or more HIV antigens and chitosan, or a derivative thereof, to the subject;
thereby treating or preventing human immunodeficiency virus.

6. The method of claim 5, wherein the HIV antigen is selected from the group consisting of gp120, p24, gp41, p17, HIV gag protein, HIV RT protein, HIV Nef protein, HIV pol protein, HIV env protein, HIV Tat protein.

7. A method for treating or preventing cancer in a subject, the method comprising the step of:
i) administering a depot composition comprising one or more cancer antigens
and chitosan, or a derivative thereof, to the subject;
thereby treating or preventing cancer.

8-9. (canceled)

10. A method for treating or preventing malaria in a subject, the method comprising the step of:
i) administering a depot composition comprising one or more malaria antigens
and chitosan, or a derivative thereof to the subject;
thereby treating or preventing malaria.

11. The method of claim 10, wherein the malaria antigen is selected from the group consisting of MSP 1, MSP 1-42, MSP 1-19, MSP1, MSP2, MSP3, MSP4, MSP5, AMA1, PfEMP1, RESA, RAP1, RAP2, Pf332, Pf155/RESA, ME-TRAP, CS, merozoite protein, parasitized red blood cells, protozoa, protozoa extracts, protozoa fragments, and inactivated protozoa.

12. A method for treating or preventing hepatitis in a subject, the method comprising the step of:
i) administering a depot composition comprising one or more hepatitis antigens
and chitosan, or a derivative thereof to the subject;
thereby treating or preventing hepatitis.

13. (canceled)

14. A method for treating or preventing influenza in a subject, the method comprising the step of:
i) administering a depot composition comprising one or more influenza antigens
and chitosan, or a derivative thereof to the subject;
thereby treating or preventing influenza.

15. The method of claim 14, wherein the one or more antigens are selected from the group consisting of HA, NA, H5N1, H1N1, H2N2, H3N2, H7N7, H1N2, H9N2, H7N2, H7N3, H10N7, and HPAI A (H5N1).

16. A method for increasing an immune response in a subject, the method comprising the step of:
i) administering a depot composition comprising one or more cytokines and
chitosan, or a derivative thereof to a subject;
thereby increasing an immune response in a subject.

17-24. (canceled)

25. A method for increasing an immune response in a subject, the method comprising the step of:
i) administering a depot composition comprising one or more cytokines, one or
more antigens and chitosan, or a derivative thereof to a subject;
thereby increasing an immune response in a subject.

26-31. (canceled)

32. A method for increasing a cell mediated immune response in a subject, the method comprising the steps of:
i) mixing particles, comprised of chitosan or a derivative thereof, containing one or more antigens; and
ii) delivering the antigen-containing chitosan-based particles to a subject;
thereby producing an immune response in the subject.

33. The method of claim 32, wherein the particles are microparticles.

34. The method of claim 32, wherein the particles are nanoparticles.

35-41. (canceled)

42. A method for treating or preventing human immunodeficiency virus in a subject, or treating or preventing malaria in a subject, treating or preventing hepatitis in a subject, treating or preventing influenza, the method comprising the steps of:
i) mixing nanoparticles or microparticles, comprised of chitosan or a derivative thereof, containing one or more antigens; and
ii) delivering the antigen-containing chitosan-based nanoparticles or microparticles to a subject;
thereby treating or preventing human immunodeficiency virus in the subject, treating or preventing malaria in the subject, treating or preventing hepatitis in the subject, or treating or preventing influenza in the subject.

43-53. (canceled)

54. A method for increasing cell mediated immune response in a subject, the method comprising the steps of:
mixing one or more antigens with chitosan, or a derivative thereof;
administering the chitosan antigen mixture to a subject; and
administering one or more cytokines to the subject;
thereby increasing cell mediated immune response in the subject.

55-58. (canceled)

59. A method for increasing cell mediated immune response in a subject, the method comprising the steps of:
mixing one or more cytokines with chitosan, or a derivative thereof;
administering the chitosan cytokine mixture to a subject;
administering one or more additional vaccines in the subject;
thereby increasing cell mediated immune response in the subject.

60-68. (canceled)

69. A method for treating or preventing cancer in a subject, the method comprising the step of:
i) administering a depot composition comprising one or more small molecule inhibitors and chitosan, or a derivative thereof, or a depot composition comprising one or antibodies, or fragments thereof, and chitosan, or a derivative thereof, to the subject;
thereby treating or preventing cancer.

70-75. (canceled)

76. A depot composition for administration in a subject, the depot composition comprising one or more cytokines and chitosan, or a derivative thereof, or one or more antigens and chitosan, or a derivative thereof.

77-124. (canceled)

125. A kit comprising a chitosan antigen depot, together with instructions for use.

126-133. (canceled)

134. A method for making a vaccine depot composition comprising mixing chitosan, or a derivative thereof, with one or more antigens, thereby making a vaccine depot composition.

135-140. (canceled)
